(19) United States
(12) Patent Application Publication
(10) Pub. No.: US 2014/0147413 A1
(43) Pub. Date: May 29, 2014

(54) Therapies That Target Autoimmunity For Treating Glaucoma And Optic Neuropathy

(71) Applicants: Dong Feng Chen, Newtonville, MA (US); Jianzhu Chen, Lexington, MA (US); Huihui Chen, Hunan (CN)

(72) Inventors: Dong Feng Chen, Newtonville, MA (US); Jianzhu Chen, Lexington, MA (US); Huihui Chen, Hunan (CN)

(21) Appl. No.: 14/002,036

(22) PCT Filed: Feb. 28, 2012
(86) PCT No.: PCT/US2012/027036

§ 371 Date: Feb. 13, 2014

Related U.S. Application Data
(60) Provisional application No. 61/447,379, filed on Feb. 28, 2011.
(60) Provisional application No. 61/481,400, filed on May 2, 2011.

(51) Int. Cl. G01N 33/68; G01N 33/569; A61K 45/06; A61K 39/395; A61K 31/436

(52) U.S. Cl. CPC G01N 33/6854; A61K 39/3955; A61K 31/436; A61K 45/06; G01N 33/56972
USPC 424/85.4; 424/173.1; 424/154.1; 514/291; 435/7.92; 435/7.24

(57) ABSTRACT

The present invention comprises a composition with means to inhibit an autoimmune response and methods for using this composition to treat glaucoma and optic neuropathy.

### RELATED APPLICATIONS

This application claims the benefit of priority under 35 U.S.C. §119(e) to U.S. Provisional Application No. 61/447,379, filed Feb. 28, 2011 and to U.S. Provisional Application No. 61/481,400, filed May 5, 2011, each of which is incorporated herein by reference in its entirety.

### STATEMENT AS TO FEDERALLY SPONSORED RESEARCH

This invention was funded in part by the U.S. Government under grant number R01-EY017641, awarded by the National Institutes of Health and the National Eye Institute. The Government has certain rights in the invention.

### FIELD OF THE INVENTION

This invention relates generally to the field of ophthalmology.

### BACKGROUND OF THE INVENTION

World-wide, glaucoma is the second leading cause of irreversible blindness, affecting one in two hundred people aged fifty and younger, and one in ten people over the age of eighty. A primary risk factor for glaucoma is elevated intraocular pressure (TOP), which contributes to significant optic nerve damage and loss of retinal ganglion cells (RGCs) in a characteristic pattern of optic neuropathy. Left untreated, glaucoma leads to permanent damage of the optic nerve and visual field loss, which often progresses to irreversible blindness. Prior to the invention described herein, treatment of glaucoma was primarily directed at lowering intraocular pressure using eye drops or surgical interventions, which slows, but does not stop the progression of vision loss. As such, there is a pressing need for new strategies for the early diagnosis and treatment of glaucoma.

### SUMMARY OF THE INVENTION

The present invention is based in part on the discovery that autoimmune CD4+ T cells responses to heat shock proteins, e.g., heat shock protein 27 (hsp27) and/or heat shock protein 60 (hsp60) mediate progressive neurodegeneration in ocular disorders such as glaucoma and optic neuropathy. For example, an autoimmune response initiated by elevated intraocular pressure (TOP) is a key component in causing progressive retinal ganglion cell (RGC) and axonal degeneration in glaucoma.

The invention provides a method for the early diagnosis and evaluation of treatment efficacy of an heat shock protein (hsp)-mediated (e.g., hsp27 or hsp60) ocular neurodegenerative condition by detecting auto-antigen-reactive T cells or auto-antigen-antibodies. The invention also provides therapeutic treatment of glaucoma, anterior ischemic optic neuropathy (AION), and optic nerve trauma by inhibiting an autoimmune response triggered by elevated TOP, ischemia, trauma or other injury or insult in a subject.

The conditions to be treated are characterized by an increase in auto-reactive T cells or antibodies, e.g., specific for hsps, compared to normal control levels of T cells or antibodies or an increased level of the heat shock proteins themselves. The subject is preferably a mammal in need of such treatment. The mammal can be, e.g., any mammal, e.g., a human, a primate, a mouse, a rat, a dog, a cat, a cow, a horse, or a pig. In a preferred embodiment, the mammal is a human.

Specifically, described herein are methods for detecting, inhibiting, or reducing the severity of glaucoma, AION or optic nerve damage as a result of trauma or other injury or insult. For example, the method inhibits or reduces the severity of primary open angle glaucoma, closed angle glaucoma, secondary glaucoma, or congenital glaucoma. First, a subject characterized as suffering from glaucoma is identified. Optionally, the identifying step comprises detection of a sign or symptom selected from the group consisting of loss of peripheral vision, optic nerve cupping, thinning of the nerve fiber layer, severe unilateral eye pain, cloudy vision, nausea and vomiting, red eye, swollen eye, eye enlargement, light sensitivity, and tearing.

In some cases, the subject has an elevated TOP as compared to a “normal level” or “control level.” As used herein, the term “normal level” or “control level” is meant to describe a value within an acceptable range of values that one of ordinary skill in the art and/or a medical professional would expect a healthy subject of similar physical characteristics and medical history to have. For example, normal TOP is defined as TOP in the range of 10 mm Hg to 21 mm Hg. Alternatively, the subject has normal intraocular pressure with optic nerve cupping and visual field loss characteristic of glaucoma.

A composition comprising an immunosuppressant agent is administered to an ocular or adnexal tissue of a subject identified as having glaucoma, thereby inhibiting or reducing the severity of glaucoma. Suitable immunosuppressant agents include a polynucleotide, a polypeptide, an antibody, and a small molecule, or conjugates thereof. Suitable immunosuppressant agents include antibodies, small molecules, glucocorticoids, cytostatics, inhibitors of hsp27, and inhibitors of hsp60. Other immunosuppressant agents include cyclosporine, FK506, tacrolimus, rapamycin, interferons, opiods, tumor necrosis factor-alpha binding protein, mycophenolate, fingolimod, and myriocin. In one aspect, the immunosuppressive agent is an antibody specific for CD3, e.g., muromonab-CD3 antibody (Orthoclone OKT3).

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

The method comprises inhibiting or reducing the severity of secondary phase neuronal damage (i.e., progressive glaucomatous neurodegeneration). For example, the method comprises inhibiting or reducing the severity of RGC damage or axonal damage.

Candidate agents are screened to identify potential inhibitors of the autoimmune response involved in RGC and optic nerve degeneration. For example, general immune suppressors and specific inhibitors of T cell or B cell-mediated autoimmunity are useful immunosuppressive agents for inhibiting or reducing the severity of glaucoma or vision loss.

Suitable inhibitors of T cell-mediated autoimmunity include dantrolene, FUT-175, a Kv1.3 inhibitor, a phosphodiesterase-3 inhibitor, a phosphodiesterase-4 inhibitor, anti-TNF alpha, anti-IFN-γ, an antibody that depletes T cells, or a molecule that suppresses T cell function without eliminating T cells. For example, the antibody that depletes T cells is an anti-CD4 antibody, an anti-CD3 antibody, or an anti-CD52 antibody (or any other antibodies that deplete T cells or neutralize effector molecules secreted by T cells or regulate autoimmune responses). For example, the inhibitor of T cell-mediated autoimmunity is an inhibitor of CD4+ T cell-mediated autoimmunity to hsp27 or hsp60.

Examples of categories of therapeutics provided herein include: 1) small molecular weight immunosuppressants; 2) biologics (e.g., antibodies) that suppress autoimmune responses, such as antibodies that deplete CD4⁺ T cells or antibodies that neutralize effector molecules of T cells or molecules that regulate autoimmune responses (without depleting T cells or neutralizing effector molecules); 3) molecules that inhibit/target hsp's. To prevent general immune suppression, these molecules are preferably delivered locally in the eye.

Optionally, the methods further comprise administering an agent that reduces intraocular pressure. Suitable agents that reduce intraocular pressure include pilocarpine, timolol, acetazolamide, clonidine, ecothiopate, carteolol, dorzolamide, apraclonidine, latanoprost, and bimatoprost. In some aspects, the method further comprises administering an inhibitor of hsp27 or hsp60.

The method for inhibiting or reducing the severity of glaucoma optionally comprises combinatorial therapy comprising the administration of an agent that reduces intraocular pressure, an immunosuppressive agent, an hsp inhibitor, and a modulator of autoreactive T cells.

The form of said composition is a solid, a paste, an ointment, a gel, a liquid, an aerosol, a mist, a polymer, a film, an emulsion, or a suspension. Optionally, the composition is administered topically. In some cases, the method does not comprise systemic administration or substantial dissemination to non-ocular tissue. Alternatively, the method does comprise systemic administration or substantial dissemination to non-ocular tissue. The invention also provides methods of inducing tolerance for specific autoimmune responses, such as that specific for small hsps. In one example, such agents or combinations of agents are administered after surgery.

Also described herein are methods for diagnosing an hsp-mediated ocular neurodegenerative condition, identifying a patient that has or is at risk of developing an hsp-mediated ocular neurodegenerative condition, and evaluating disease progression and treatment efficacy by detecting the levels of auto-antigen-specific antibodies or auto-antigen-specific T cells in a test sample from a subject. In one aspect, the subject comprises RGC damage or axonal damage. Preferably, the condition is diagnosed early (i.e., prior to vision loss). For example, T cells and/or antibodies are detected in peripheral blood (i.e., cells, serum, or plasma) obtained from the subject.

Specifically, the invention provides methods of diagnosing an hsp-mediated ocular neurodegenerative condition in a subject by providing a test sample from a subject and detecting auto-antigen antibodies or auto-antigen-specific T cells in the test sample. Suitable test samples include biological fluids selected from the group consisting of whole blood, serum, plasma, vitreous humor, and aqueous humor. The levels of the auto-antigen antibodies or auto-antigen-specific T cells in the test sample are compared to a control level of the antibodies or T cells. For example, the control level is obtained from age-matched healthy individuals. A higher level of the antibodies or T cells compared to the control level is indicative of the condition, thereby diagnosing the condition in the subject. Preferably, the auto-antigen is selected from the group consisting of hsp27 (also known as heat shock protein beta-1 (hspB1)), hsp60, alpha-A-crystallin, and alpha-B-crystallin. The condition is glaucoma, AION, or optic nerve damage.

Methods for the therapeutic treatment of optic neuropathy caused by ischemia and trauma are described herein. Such methods are carried out by inhibiting autoimmune responses triggered by initial acute injury. Candidate agents are screened to identify potential inhibitors of the autoimmune response involved in degradation of the central nervous system, e.g., nerve fibers and neurons. The invention also provides methods of inducing tolerance for specific autoimmune responses, such as that specific for small hsps.

Specifically, described herein are methods for inhibiting or reducing the severity of optic neuropathy, e.g., AION. For example, the method comprises inhibiting or reducing the severity of secondary phase neuronal damage associated with optic neuropathy. In one aspect, the method comprises inhibiting or reducing the severity of RGC damage or axonal damage. First, a subject characterized as suffering from ischemia or trauma-induced optic neuropathy is identified. An immunosuppressant agent is locally administered to an ocular or adnexal tissue of a subject, thereby inhibiting or reducing the severity of secondary phase neuronal damage associated with optic neuropathy. For example, the immunosuppressant agent is muromonuab-CD3 antibody OKT3, or fragments of such antibodies, so long as they exhibit the desired biological activity.

Also included in the invention are chimeric antibodies, such as humanized antibodies. Generally, a humanized antibody has one or more amino acid residues introduced into it from a source that is non-human. Humanization can be performed, for example, using methods described in the art, by substituting at least a portion of a rodent complementarity-determining region for the corresponding regions of a human antibody.

The term “antibody” or “immunoglobulin” is intended to encompass both polyclonal and monoclonal antibodies. The preferred antibody is a monoclonal antibody reactive with the antigen. The term “antibody” is also intended to encompass mixtures of more than one antibody reactive with the antigen (e.g., a cocktail of different types of monoclonal antibodies reactive with the antigen). The term “antibody” is further intended to encompass whole antibodies, biologically functional fragments thereof, single-chain antibodies, and genetically altered antibodies such as chimeric antibodies comprising portions from more than one species, bifunctional antibodies, antibody conjugates, humanized and human antibodies. Biologically functional antibody fragments, which can also be used, are those peptide fragments derived from an antibody that are sufficient for binding to the antigen. “Antibody” as used herein is meant to include the entire antibody as well as any antibody fragments (e.g., F(ab′)₂, Fab′, Fab, Fv) capable of binding the epitope, antigen or antigenic fragment of interest.

The method optionally includes administering an inhibitor of T cell or B cell-mediated autoimmunity. For example, an inhibitor of T cell-mediated autoimmunity is an inhibitor of CD4+ T cell-mediated autoimmunity to hsp27 or hsp60. In one aspect, the method comprises administering an agent that reduces intraocular pressure. The method optionally further comprises administering an inhibitor of hsp27 or hsp60.

Methods of diagnosing or evaluating treatment efficacy of optic neuropathy, e.g., AION, glaucoma, or optic nerve damage in a subject are carried out by providing a test sample from a subject and detecting auto-antigen antibodies or auto-antigen-specific T cells in the test sample. The test sample is obtained from a biological fluid selected from the group consisting of whole blood, serum, plasma, vitreous humor, and aqueous humor. The levels of the auto-antigen antibodies or the auto-antigen-specific T cells in the test sample are compared to a control level of the antibodies or T cells. For example, the auto-antigen is selected from the group consisting of hsp-27, hsp-60, alpha-A-crystallin, and alpha-B-crystallin. A higher level of the antibodies or T cells compared to the control level is indicative of optic neuropathy or glaucoma in the subject. For example, the level of hsp-reactive T cells in peripheral blood is increased by at least 20%, at least 50%, 2 fold, 3 fold, 5 fold, 7 fold, or more compared to a normal control level. The subject optionally comprises RGC damage or axonal damage.

The diagnostic methods of the invention provide a solution to a long-standing problem in the field, i.e., the failure to detect the disease or disorder until overt physical impairment, e.g., vision impairment, occurs. The diagnostic methods described herein detect neurodegeneration in glaucoma and ischemic optic neuropathy at an early stage. Early diagnosis permits early intervention to avoid the slow debilitating symptoms.

The invention also provides kits for the treatment and diagnosis of glaucoma and other ocular disorders utilizing the methods described herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Polynucleotides, polypeptides, or other agents are purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage.

The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause.

By the terms “effective amount” and “therapeutically effective amount” of a formulation or formulation component is meant a nontoxic but sufficient amount of the formulation or component to provide the desired effect.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1D are a series of a line graph, a series of photomicrographs, and bar charts demonstrating that transiently elevated IOP induces progressive axon and RGC degeneration. IOP was induced by anterior chamber injection of polystyrene microbeads.

FIG. 1A is a line graph showing a comparison of IOP levels in mice received anterior chamber injection of microbeads (n=18) or PBS (n=6). IOP was measured every other day starting from day 0 before the injection. FIG. 1B is a series of photomicrographs showing electron microscopy (EM) and immunofluorescence (Tuj1) analysis of axon and RGC loss in optic nerve sections and retinal flat-mounts in mice 2 months post PBS or microbead injection (High IOP). Retinal flat-mounts were immunolabeled with a primary antibody specific to an RGC specific marker, Tuj1-1, followed by an Alexa Fluor 488-conjugated secondary antibody. Scale bars: 5 μm (EM); 25 μm (anti-Tuj1). FIG. 1C and FIG. 1D are bar charts showing the quantification of axon (C) and RGC (D) loss at various time points after microbead injection. Mice were sacrificed at 0, 2, 4, and 8 weeks after microbead injection (n=6/group) or at 8 weeks after PBS injection (PBS; n=6). Loss of axon and RGC (mean±S.D.) is presented as percentage of axon or RGC counts from optic nerve sections or retinal flat-mounts of microbead-injected eyes, respectively, over that of the uninjected contralateral eyes. For comparison, the kinetics of IOP elevation in microbeads injected eyes is reproduced from 1a. *P<0.05 between pairs of comparison.

FIG. 2A-FIG. 2B are a series of photomicrographs and a bar chart, respectively, showing that IOP elevation induces T cell infiltration and complement deposition in the retina. FIG. 2A is a series of photomicrographs showing immunofluorescent staining for CD3 and C1q. Retinal flat-mounts from mice at 3 weeks after microbead injection were double stained with Tuj1 (red) and anti-CD3 (green) or anti-C1q (green) and then counter-stained with a nuclear marker 4′,6-diamidino-2-phenylindole (DAPI; blue). Arrows point to CD3- or C1q-stained cells. Note the association of infiltrated T cells with RGC axons and C1q deposition on RGC bodies. Scale bar: 10 μm. FIG. 2B is a bar chart showing quantification of T cell infiltration in retinal flat-mounts of B6 mice at 1, 2, 3 and 4 weeks post injection of microbeads (High IOP) or 8 month old DBA/2J mice (n=6/group). Retinal flat-mounts of the uninjected contralateral eyes (Cont) or eyes 2 weeks after receiving anterior chamber injection of PBS (PBS) were used as controls.

FIG. 3A-FIG. 3F are a series of photomicrographs and bar charts demonstrating that T cell deficiency attenuates elevated IOP-induced secondary glaucomatous axon and RGC degeneration, and transfer of T cells from high IOP mice restores the secondary neurodegeneration in T cell deficient mice. C57BL/6 (B6), Rag1−/−, TCRβ−/−, and Igh6−/− mice were injected with microbeads in the anterior chamber of one eye and analyzed for axon and RGC loss at 2 and 8 weeks post injection. FIG. 3A is a series of representative electron micrographs of optic nerve sections and immunofluorescent staining of retinal flat-mounts 8 weeks post microbead (High IOP) or PBS (PBS) injection. Scale bars: 2 μm (EM); 25 μm (Tuj1). FIGS. 3B and 3C are bar charts showing the comparison of axon and RGC loss (mean±S.D.) among various types of mice between 2 (dark bar) and 8 (gray bar) weeks post microbead injection (n=6/group). *P<0.05 and NS (not significant) refer to comparisons of the same type of mice between 2 and 8 weeks post injection; @P<0.05 refers to comparisons between B6 and mutant mice at the corresponding time points of microbead injection. For FIGS. 3D-3F, CD4+ T cells were isolated from the spleen of wild-type mice 2 weeks after anterior chamber injection of microbeads or PBS and injected into Rag1−/− mice 2 weeks after induction of IOP elevation. Recipient mice were sacrificed 2 weeks after cell transfer and analyzed for axons and RGCs in optic nerve sections and retinal flat-mounts. FIG. 3D is a series of representative photomicrographs showing immunofluorescent staining of retinal flat-mounts 2 weeks post cell injection (or 4 weeks post microbead injection). The retinal flat-mounts were triple-labeled by anti-CD4+ (green) and Tuj1 (red) antibodies and DAPI (blue). PBS, Rag1−/− mice transferred with CD4+ T cells from PBS injected B6 mice; High IOP, Rag1−/− mice transferred with CD4+ T cells from microbead injected B6 mice. Scale bar: 30 μm (left panel); 10 μm (right panel). FIG. 3E and FIG. 3F are bar charts showing quantification (mean±S.D.) of axon and RGC loss in Rag1−/− mice that received no cell transfer (Rag1−/−), or CD4+ T cell transfer from B6 mice with high IOP (High IOP) or PBS injection (PBS; n=5/group). * indicates p value of <0.05 compared to the Rag1−/− group.

FIG. 4A-FIG. 4C are a series of photomicrographs, an immunoblot, and a bar chart demonstrating that induction of hsp27 expression in RGCs and serum hsp27 autoantibodies following elevation of IOP. B6 mice 1, 2, 4, and 8 weeks after injection with microbeads or 2 weeks after PBS injection (to serve as controls) were analyzed for hsp27 and hsp60 expression by immunofluorescence staining or Western blotting of retinas and for hsp27- and hsp60-specific antibodies in the sera. FIG. 4A is a series of photomicrographs showing representative immunofluorescence staining of retinal flat-mounts from mice 4 weeks after injection of microbeads (high IOP) or PBS (PBS): anti-hsp27 (green) and Tuj1 (red). Scale bar: 10 μm. FIG. 4B is a photograph of a Western blot analysis of hsp27 and hsp60 expression in the retinas of mice at different time points after microbead (High IOP) or at 2 weeks after PBS (PBS) injection. FIG. 4C is a bar chart showing ELISA quantification of autoantibodies specific for hsp27 or hsp60 in the sera of mice at different time points after microbead (High IOP) or PBS (PBS) injection (n=6/group). *P<0.05 as compared to PBS injected group.

FIG. 5A-FIG. 5E are a series of photomicrographs and bar charts demonstrating that elevated IOP induces hsp27 specific T cell responses. One, 2 or 8 weeks after injection of microbeads, mice were injected intradermally in the ears with recombinant hsp27, MBP or IRBP. Ear thickness was measured 24 hrs later. T cell infiltration in the ear was assayed by anti-CD4 immunofluorescence, and IFN-γ secreting T cells in the spleen were assayed by ELISPOT. FIG. 5A is a photomicrograph showing the comparison of abundance of CD4+ T cells in the ear section of B6 mice with an anterior chamber injection of PBS (B6 PBS) or microbeads (B6 high IOP) and Rag1−/− mice with microbead injection (Rag1−/− high IOP). Scale bar: 50 μm. FIG. 5B is a bar chart showing the comparison of ear thickness changes in B6 mice with a normal IOP (PBS), B6 mice 1, 2, and 8 weeks (w) after anterior chamber injection of microbeads or B6 mice that were injected with control antigens, IRBP or MBP, 2 weeks after microbead injection. FIG. 5C is a bar chart showing the comparison of ear thickness changes in B6, Rag1−/− and TCRβ−/− mice 2 weeks after microbead injection. FIG. 5D is a bar chart showing quantification of ELISPOT assays: Splenocytes from B6 mice with a normal IOP (PBS) or B6 mice 1, 2, and 8 weeks after microbead injection or Rag1−/− and TCRβ/− 2 weeks post microbead injection were stimulated by hsp27 or MBP in vitro. Secretion of IFN-γ was detected by ELISPOT. FIG. 5E is a bar chart showing the comparison of frequency of IFN-γ secreting T cells in splenocytes from 11 (11) and 40 (40) week old DBA/2J mice and 40 week old B6 mice (B6). Mean±S.D. (n=6/group) was shown for b-e. *P<0.05; **P<0.001 as compared to the respective control groups.

FIG. 6A-FIG. 6F are a series of bar charts showing the induction of axon and RGC damage following adoptive transfer of hsp27 responsive T cells and increased hsp27 and hsp60 responsive T cells and autoantibodies in glaucoma patients. FIG. 6A is a bar chart showing the comparison of DTH responses (ear thickness) between hsp27 and ovalbumin immunized mice. B6 mice were immunized with hsp27 (hsp27) or ovalbumin (Ova) in IFA. Two weeks later, mice were injected intradermally with hsp27, and ear thickness was measured 24 hrs later (n=6/group). FIG. 6B is a bar chart showing the comparison of the frequencies of IFN-γ secreting cells in the spleen of hsp27 and Ova immunized mice. Two weeks after immunization, CD4+ T cells were isolated from spleen of immunized mice and stimulated in vitro with hsp27. The frequencies of IFN-γ secreting cells were quantified by ELISPOT (n=6/group). FIG. 6C and FIG. 6D are bar charts showing the effect of CD4+ T cell transfer on loss of axons and RGCs in recipient mice. Two weeks after hsp27 or Ova immunization, CD4+ T cells were purified from spleen and adoptively transferred to B6 mice that had been induced to develop high IOP for 2 weeks. None, B6 recipients without cell transfer; Ova and hsp27, B6 recipients transferred with CD4+ T cells from Ova or hsp27 immunized mice, respectively (n=6/group). *P<0.05 as compared mice without cell transfer. In FIG. 6E and FIG. 6F, the peripheral blood from glaucoma patients and age-matched healthy controls were obtained. Frequencies of hsp27 and hsp60 responsive T cells were assayed by ELISPOT, and hsp27 and hsp60-specific antibodies were quantified by ELISA. Comparison of frequencies of hsp27 and hsp60 responding T cells (FIG. 6E) and hsp27 and hsp60-specific antibodies (FIG. 6F) between glaucoma patients (Patients; n=11) and healthy individuals (Normal; n=8). *P<0.05 between patients and healthy individuals.

FIG. 7 is a line graph showing that anterior chamber injection of PBS does not affect the level of IOP. The IOP of mice who received an anterior chamber injection of PBS (n=8) remained at the baseline level as compared to uninjected control eyes (n=6). IOP were measured every other day staring at day 0 before the injection.

FIG. 8 is a line graph showing that T cell and/or B cell deficiency does not affect IOP profile. Mice deficient in Rag^{1-/-}, TCRβ^{-/-}, or Igh6^{-/-} showed a similar baseline level (n=8/group) or kinetics of IOPs after receiving an anterior chamber injection of microbeads (MB; n=8/group). IOP were measured every other day starting at day 0 before injection.

FIG. 9 is a bar chart showing that suppressing autoimmunity using immuno-deficient mice or immune suppressor promotes RGC survival after optic neuropathy. Optic never crush injury was performed in wild-type (wt) mice that received daily injection of PBS (wt) or rapamycin (Rapam) as well as in Rag^{1-/-} (Rag1) and TCRβ^{-/-} (TCRβ) mice. Animals were sacrificed 4 weeks post operation, and percentage of RGC loss was assessed. (*P<0.01 as compared to the normal subject group).

FIG. 10 is a schematic representation of ischemic optic neuropathy, which results in the elevation of intraocular pressure.

FIG. 11A-FIG. 11B are a series of photomicrographs and a bar chart demonstrating that acute AION in mice induced progressive axon and RGC degeneration that lasted over 4 weeks (indicating that acute injury triggers a secondary event contributing to the progressive neurodegeneration). FIG. 11A is a series of photomicrographs showing representative electron microscopy (EM) and immunofluorescence (Tuj-1) analysis of axon and RGC loss in optic nerve sections and retinal flat-mounts in mice 7 and 28 days following induction of acute AION by elevation of IOP to 100 mmHg for 1 hour. Retinal flat-mounts were immunolabeled with a primary antibody for an RGC specific marker, Tuj 1-1, followed by an AlexaFluor 488-conjugated secondary antibody. Scale bars: 2 μm (EM); 25 μm (Tuj1). FIG. 11B is a bar chart showing the quantification of RGC loss at various time points after the induction of AION. Mice were sacrificed at 0, 3, 7, 28, and 56 days after AION (n=6/group) or at 28 days after sham operation (n=6). Loss of RGCs (mean±S.D.) is presented as percentage of RGC counts from retinal flat-mounts of injured eyes relative to that of the uninjured contralateral eyes. *P<0.05, **P<0.001 by two tailed student t test.

FIG. 12 is a series of photomicrographs showing the induction of hsp27 and hsp60 expression in RGCs following AION. The figure shows representative photomicrographs of B6 wild-type mice at 1 and 4 weeks after induction of AION or 4 weeks after sham operation that were immunolabeled for hsp27 and hsp60. The data indicate upregulation of hsp27 and hsp60 in the retina following AION as compared to the wild-type control retina. Scale bar: 15 μm.

FIG. 13A-FIG. 13C are a series of photomicrographs and bar charts demonstrating that AION induces CD4+ T cell infiltration into the retina. FIG. 13A is a series of photomicrographs showing double immunolabeling of CD4 (green) and Tuj1 (red) in retinal flat-mounts taken from mice at 2 weeks after the induction of AION. The retina flat-mount was also counter-stained with nuclear marker 4′,6-diamidino-2-phenylindole (DAPI; blue). Scale bar: 10 μm. FIG. 13B is a bar chart showing the quantification of T cell infiltration into retinal flat-mounts of B6 wild-type mice at 3, 7, 14 and 28 days (D) following AION or from 28 days of sham operated mice (n=6/group). *P<0.05 as compared to sham group. FIG. 13C is a bar chart showing the results of RT-PCR that detect 4 types of T cell markers, IFNγ (TH1), interleukin 4 (IL4; TH2), IL17 (TH17) and TNFα (Treg), expression in the injury retina at different time points after AION. The results show a significant increase of IFNγ after AION as compared to sham control, indicating infiltrated T cells are predominantly TH1.

FIG. 14 is a bar chart showing that acute AION induces hsp27 and hsp60-specific T cell responses. The figure shows quantification of ELISPOT assays that assessed IFN-γ secreting T cells in the lymph node taken from mice at 3, 7 and 28 days after AION. Lymphocytes taken from these mice were stimulated by hsp27, hsp60 or ova (as control stimulation) in vitro. Secretion of IFN-γ was detected by ELISPOT. *P<0.05 as compared to the respective sham groups.

FIG. 15A-FIG. 15C are a series of photographs, a cell plot, and a bar chart showing that acute AION induces CD11b+ cell migration to the draining lymph node and active T cell. FIG. 15A shows representative draining lymph nodes taken from mice at 7, 14 and 28 days post AION induction or from sham-operated group. FIG. 15B is a chart showing representative flow analysis of CD4+/IFNγ+ cells, and demonstrates an increase of CD4+/IFNγ+ cells in the AION mice as compared to the sham group. FIG. 15C shows relative expression of IFNγ+ cells detected at draining lymph node at different time point post AION.

FIG. 16A-FIG. 16C are a series of bar charts demonstrating that T cell deficiency attenuates elevated ischemia-induced secondary axon and RGC degeneration, and transfer of T cells from AION mice restores secondary neurodegeneration in T cell deficient mice. C57BL/6 (B6), Rag1−/− and TCKO mice were induced ischemia and analyzed for axon and RGC loss at 1 and 4 weeks post injury. FIG. 16A shows a comparison of RGC loss (mean±S.D.) among C57BL/6 and Rag1−/− mice between 1 and 4 weeks post ischemia or sham operation at 4 weeks (n=6/group). *P<0.05 N p>0.05. FIG. 16B shows a comparison of RGC loss (mean±S.D.) among C57BL/6 and TCKO mice between 1 and 4 weeks post ischemia or sham operation at 4 weeks (n=6/group). *P<0.05. CD4+ T cells were isolated from the spleen of wild-type ischemia mice and sham group at 2 weeks after injury, and injected into Rag1−/− mice 2 weeks after induction of ischemia. Recipient mice were sacrificed 2 weeks after cell transfer and analyzed for RGCs in retinal flat-mounts. FIG. 16C shows the quantification (mean±S.D.) of RGC loss in Rag1−/− mice that induced ischemia for 4 weeks or received CD4+ T cell transfer from B6 mice with ischemia group or sham group (n=6/group). * indicates p value of <0.05 compared to sham group.

FIG. 17A-FIG. 17B is a series of photomicrographs and a bar chart demonstrating that OKT3 antibody administration resulted in a neuroprotective effect for AION. OKT3 antibody was injected into the vitreous of ischemia WT mice at 3, 7, and 14 days post injury. Injection of IgG isotype served as the control. All of the recipient mice were sacrificed at 4 weeks after AION. FIG. 17A shows representative photomicrographs of immunofluorescent labeled RGCs (Tuj-1) in retinal flat-mounts of wild-type mice with AION that received no treatment, control IgG, or OKT3 antibody treatment. FIG. 17B shows the quantification (mean±S.D.) of RGC loss in mice (n=6/group). * indicates p value of <0.05.

FIG. 18 is a line graph showing the functional rescue of retinal activity by OKT3 treatment after AION. Specifically, the figure shows a comparison of electroretinograma and b wave length (mean±S.D.) between the sham group, IgG isotypeinjected ischemia group and OKT3 antibody injected ischemia group at different intensity of light stimulation. *P<0.05 refers to comparisons between IgG isotype-injected group and sham group. #P<0.05 refers to comparisons between IgG isotype-injected group and OKT3 antibody-injected group (n=6/group).

### DETAILED DESCRIPTION

The present invention provides compositions and methods for diagnosing, treating and/or preventing ophthalmic or ocular disorders, diseases or conditions, and compositions and methods for treating or preventing ophthalmic or ocular conditions and disorders in a subject in need thereof. Specifically, the present invention is based in part on the discovery that an autoimmune response initiated by elevated IOP, trauma, ischemia, or other injury, and insult is the key component in causing progressive retinal ganglion cell (RGC) or other neuron and axonal degeneration associated with glaucoma, AION, or optic nerve trauma. As described herein, specific inhibition of the autoimmune response inhibits or reduces the severity of glaucoma symptoms or neurodegeneration associated with optic neuropathy. Also described herein are methods for diagnosing glaucoma, identifying a patient at risk of developing glaucoma, and evaluating disease progression and treatment efficacy by detecting elevated levels of auto-antigen antibodies or auto-antigen-specific T cells in a test sample from a subject. The subject is preferably a mammal in need of such treatment. The mammal can be, e.g., any mammal, e.g., a human, a primate, a mouse, a rat, a dog, a cat, a cow, a horse, or a pig. In a preferred embodiment, the mammal is a human.

Prior to the invention described herein, the treatment of glaucoma was primarily directed at lowering intraocular pressure using eye drops or surgical intervention. However, lowering intraocular pressure slows, but does not stop the progression of vision loss. Thus, prior to the invention described herein, therapies involving lowering intraocular pressure for ischemic optic neuropathy or optic nerve trauma were ineffective.

Described herein are results that demonstrate that the pathogenesis of glaucoma, optic nerve trauma, and AION features characteristic adaptive immune responses that generate and perpetuate secondary neurodegeneration. The results presented below also demonstrate that functional deficiency of T cells attenuated glaucomatous neurodegeneration, and adoptive transfer of CD4 T cells isolated from glaucoma mice or hsp27 specific T cells restored secondary neurodegeneration in mice deficient for T cell functions. Moreover, described herein are results demonstrating that hsp27 and hsp60 specific T cells were used as diagnostic markers for RGC damage in glaucoma and AION.

### Glaucoma

Glaucoma is the most prevalent neurodegenerative disorder and the leading cause of irreversible blindness. Elevated IOP (i.e., the fluid pressure inside the eye) is a major risk factor for primary open angle glaucoma, but prior to the invention described herein, its exact role in the disease was unclear. Earlier treatment strategies were directed at lowering IOP, and were often insufficient to stop the progression of neurodegeneration and vision loss.

World-wide, glaucoma is the second leading cause of irreversible blindness, affecting one in two hundred people aged fifty and younger, and one in ten people over the age of eighty. A primary risk factor for glaucoma is elevated IOP, which can contribute to significant optic nerve damage and vision loss. A reduction in aqueous outflow facility is a major causal risk factor in elevated IOP-associated glaucoma. The main aqueous outflow pathway of the eye consists of a series of endothelial-cell-lined channels in the angle of the anterior chamber and comprises the trabecular meshwork (TM), Schlemm's canal, the collector channels, and the episcleral venous system. “Glaucoma” is a term used to describe a group of diseases of the optic nerve involving the loss of retinal ganglion cells in a characteristic pattern of optic neuropathy. Left untreated, glaucoma leads to permanent damage of the optic nerve and resultant visual field loss, which can progress to blindness. The loss of visual field often occurs gradually over a long time and may only be recognized when it is already quite advanced. Once lost, this damaged visual field can never be recovered.

As described above, ocular hypertension is the largest risk factor for glaucoma. Although elevated intraocular pressure is a significant risk factor for developing glaucoma, there is no set threshold for intraocular pressure that causes glaucoma. In some populations only 50% of patients with primary open angle glaucoma have elevated ocular pressure. Diabetics and those of African descent are three times more likely to develop primary open angle glaucoma. Higher age, thinner corneal thickness, and myopia are also risk factors for primary open angle glaucoma. People with a family history of glaucoma have about a six percent chance of developing glaucoma. Asians are prone to develop angle-closure glaucoma, and Inuit have a twenty to forty times higher risk than Caucasians of developing primary angle closure glaucoma. Women are three times more likely than men to develop acute angle-closure glaucoma due to their shallower anterior chambers. Use of steroids can also cause glaucoma.

Primary open angle glaucoma (POAG) is associated with mutations in genes at several loci. Normal tension glaucoma, which comprises one third of POAG, is associated with genetic mutations. There is increasing evidence suggesting that ocular blood flow is involved in the pathogenesis of glaucoma. Current data indicate that fluctuations in blood flow are more harmful in glaucomatous optic neuropathy than steady reductions. Unstable blood pressure and dips are linked to optic nerve head damage and correlate with visual field deterioration. A number of studies also suggest that there is a correlation, not necessarily causal, between glaucoma and systemic hypertension (i.e., high blood pressure). In normal tension glaucoma, nocturnal hypotension may play a significant role. Various rare congenital/genetic eye malformations are associated with glaucoma. Occasionally, the failure of the normal third trimester gestational atrophy of the hyaloid canal and the tunica vasculosa lentis is associated with other anomalies. Angle closure induced ocular hypertension and glaucomatous optic neuropathy may also occur with these anomalies.

Glaucoma is divided into primary open-angle glaucoma, primary closed-angle glaucoma, congenital glaucoma, secondary glaucoma, and normal tension glaucoma. Primary open angle glaucoma is caused by the slow clogging of the drainage canals, resulting in increased eye pressure. Primary close angle (acute) glaucoma causes a quick, severe, and painful rise in the pressure in the eye. Acute glaucoma in one eye presents a risk for an attack in the second eye.

Congenital glaucoma is caused by abnormal eye development. Secondary glaucoma is caused by drugs such as corticorsteroids, dilating eye drops, eye diseases such as uveitis, trauma, and vitreous hemorrhage, edema and other disease conditions such as exfoliation. Normal-tension glaucoma (NTG), also known as low tension or normal pressure glaucoma, is a form of glaucoma in which damage occurs to the optic nerve without eye pressure exceeding the normal range. In general, a “normal” pressure range is between 10-20 mm Hg.

### Anterior Ischemic Optic Neuropathy

AION is a medical condition involving loss of vision due to damage to the optic nerve from insufficient blood supply. A patient typically presents with poor vision in one eye. Vision in the eye is often obscured by a dark shadow in the area near the nose in the upper or lower half of vision. Thus, a patient with AION is identified by, inter alia, presentation with a reduced visual field. The diagnosis of AION is described in Miller N R, 1980 Bull. N.Y. Acad. Med., Vol. 56, (7): 643-654, incorporated herein by reference.

### Intraocular Pressure

IOP is maintained by the liquid aqueous humor, which is produced by the ciliary body of the eye. Aqueous humor normally does not go into the posterior segment of the eye; it is kept out of this area by the lens and the Zonule of Zinn. Instead, it stays only in the anterior segment, which is divided into the anterior and posterior chambers. While the anterior and posterior chambers are very similarly named to the anterior and posterior segments, they are not synonymous. The anterior and posterior chambers are both parts of the anterior segment. When the ciliary bodies produce the aqueous humor, it first flows into the posterior chamber (bounded by the lens and the iris). It then flows through the pupil of the iris into the anterior chamber (bounded by the iris and the cornea). From here, it flows through a structure known as the trabecular meshwork to enter the normal body circulation.

The two main mechanisms of ocular hypertension are an increased production of aqueous humor, or a decreased outflow of aqueous humor. Ocular hypertension (OHT) is intraocular pressure higher than normal in the absence of optic nerve damage or visual field loss. Current consensus in ophthalmology defines normal TOP as that between 10 mmHg and 21 mmHg. Intraocular pressure is measured with a tonometer. Elevated TOP is the most important risk factor for glaucoma, so those with ocular hypertension are frequently considered to have a greater chance of developing the condition. Intraocular pressure can increase when a patient lies down. There is evidence that some glaucoma patients (e.g., normal tension glaucoma patients) with normal TOP while sitting or standing may have intraocular pressure that is elevated enough to cause problems when they are lying down.

Differences in pressure between the two eyes are often clinically significant, and potentially associated with certain types of glaucoma, as well as iritis or retinal detachment. Because of the effect of corneal thickness and rigidity on measured value of intraocular pressure, some forms of refractive surgery (such as photorefractive keratectomy) can cause traditional intraocular pressure measurements to appear normal when in fact the pressure may be abnormally high. Intraocular pressure may become elevated due to anatomical problems, inflammation of the eye, genetic factors, as a side-effect from medication, or during exercise. Intraocular pressure usually increases with age and is genetically influenced. Hypotony, or ocular hypotony, is typically defined as intraocular pressure equal to or less than 5 mmHg. Such low intraocular pressure could indicate fluid leakage and deflation of the eyeball.

In one aspect of the invention, subjects are identified by measuring their intraocular pressure and determining if the measured intraocular pressure is elevated above normal levels. As used herein, the term “normal level” or “control level” is meant to describe value within an acceptable range of values that one of ordinary skill in the art and/or a medical professional would expect a healthy subject of similar physical characteristics and medical history to have. For example, “normal” TOP is defined as TOP in the range of 10 mm Hg to 21 mm Hg. In another aspect of the invention, subjects are identified as those individuals who are at risk for developing elevated TOP based upon non-limiting factors such as medical history (for instance, diabetes), side effects of medications, lifestyle and/or diet, medical intervention (such as surgery to the eye), trauma/injury, hormone changes, and aging. Compositions of the invention are administered to these subjects for preventative means.

Ocular hypertension is typically treated with pilocarpine (muscarinic agonist), timolol (β-receptor antagonist), acetazolamide (carbonic anhydrase inhibitor), and/or clonidine (α2-receptor agonist). Other therapeutics include ecothiopate (cholinesterase inhibitor), carteolol (β-receptor antagonist), dorzolamide (carbonic anhydrase inhibitor), apraclonidine (α-2 agonist), latanoprost (prostaglandin analogue), and bimatoprost (prostaglandin analogue). Acetazolamide is typically administered systemically; however, most ocular hypertension therapeutics are administered topically via eye drops. Other alternative therapies include medicinal cannabis.

### Ocular and Adnexal Tissues

Ocular tissues or compartments that contact the compositions comprised by the present invention include, but are not limited to, the cornea, aqueous humor, iris, and sclera. The term “adnexal” is defined in general terms as the appendages of an organ. In the present invention, adnexal defines a number of tissues or surfaces that are in immediate contact with the ocular surface but are not, by definition, comprised by the ocular surface. Exemplary adnexal tissues include, but are not limited to, the eyelids, lacrimal glands, and extraocular muscles. The compositions contact (e.g., via topical administration) the following tissues and structures within the eyelid: skin, subcutaneous tissue, orbicularis oculi, orbital septum, tarsal plates, palpebral conjuntiva, and meibomian glands. The adnexal tissues comprise all subdivisions of the lacrimal glands, including the orbital and palpebral portions, as well as all tissues contacted by these glands. Extraocular muscles belonging to this category of adnexal tissues include, but are not limited to, the superior and inferior rectus, lateral and medial rectus, and superior and inferior oblique muscles. Compositions comprised by the present invention are applied topically and contact these tissues either alone, or in combination with ocular tissues.

Administering the formulation to the eye can involve drops, injections, or implantable devices, depending on the precise nature of the formulation and the desired outcome of the administration. Specifically, a composition of the invention is delivered directly to the eye, (e.g., topical ocular drops or ointments; slow release devices such as pharmaceutical drug delivery sponges implanted in the cul-de-sac or implanted adjacent to the sclera or within the eye; and periocular, conjunctival, sub-tenons, intracameral, intravitreal, or intracanalicular injections), or systemically (e.g., orally; intravenous, subcutaneous or intramuscular injections; parenteral, dermal or nasal delivery) using techniques well known by those of ordinary skill in the art. It is further contemplated that a peptide as disclosed herein is formulated in intraocular inserts or implantable devices as described further below.

### Pharmaceutically Acceptable Carriers

The ophthalmic formulations of the invention are administered in any form suitable for ocular drug administration, e.g., dosage forms suitable for topical administration, a solution or suspension for administration as eye drops or eye washes, ointment, gel, liposomal dispersion, colloidal microparticle suspension, or the like, or in an ocular insert, e.g., in an optionally biodegradable controlled release polymeric matrix. The ocular insert is implanted in the conjunctiva, sclera, pars plana, anterior segment, or posterior segment of the eye. Implants provide for controlled release of the formulation to the ocular surface, typically sustained release over an extended time period. Additionally, in a preferred embodiment, the formulation is entirely composed of components that are naturally occurring and/or as GRAS (“Generally Regarded as Safe”) by the U.S. Food and Drug Administration.

The pharmaceutically acceptable carrier of the formulations of the invention may comprise a wide variety of non-active ingredients which are useful for formulation purposes and which do not materially affect the novel and useful properties of the invention. By a “pharmaceutically acceptable” or “ophthalmologically acceptable” component is meant a component that is not biologically or otherwise undesirable, i.e., the component may be incorporated into an ophthalmic formulation of the invention and administered topically to a patient's eye without causing any undesirable biological effects or interacting in a deleterious manner with any of the other components of the formulation composition in which it is contained. When the term “pharmaceutically acceptable” is used to refer to a component other than a pharmacologically active agent, it is implied that the component has met the required standards of toxicological and manufacturing testing or that it is included on the Inactive Ingredient Guide prepared by the U.S. Food and Drug Administration.

The compositions administered according to the present invention optionally also include various other ingredients, including but not limited to surfactants, tonicity agents, buffers, preservatives, co-solvents and viscosity building agents. In carriers that are at least partially aqueous one may employ thickeners, isotonic agents, buffering agents, and preservatives, providing that any such excipients do not interact in an adverse manner with any of the formulation's other components. It should also be noted that preservatives are not necessarily required in light of the fact that the metal complexer itself may serve as a preservative, as for example ethylenediaminetetraacetic acid (EDTA) which has been widely used as a preservative in ophthalmic formulations.

Suitable thickeners will be known to those of ordinary skill in the art of ophthalmic formulation, and include, by way of example, cellulosic polymers such as methylcellulose (MC), hydroxyethylcellulose (HEC), hydroxypropylcellulose (HPC), hydroxypropyl-methylcellulose (HPMC), and sodium carboxymethylcellulose (NaCMC), and other swellable hydrophilic polymers such as polyvinyl alcohol (PVA), hyaluronic acid or a salt thereof (e.g., sodium hyaluronate), and crosslinked acrylic acid polymers commonly referred to as “carbomers” (and available from B.F. Goodrich as Carbopol® polymers). The preferred amount of any thickener is such that a viscosity in the range of about 15 cps to 25 cps is provided, as a solution having a viscosity in the aforementioned range is generally considered optimal for both comfort and retention of the formulation in the eye. Any suitable isotonic agents and buffering agents commonly used in ophthalmic formulations may be used, providing that the osmotic pressure of the solution does not deviate from that of lachrymal fluid by more than 2-3% and that the pH of the formulation is maintained in the range of about 6.5 to about 8.0, preferably in the range of about 6.8 to about 7.8, and optimally at a pH of about 7.4. Preferred buffering agents include carbonates such as sodium and potassium bicarbonate.

Various tonicity agents are optionally employed to adjust the tonicity of the composition, preferably to that of natural tears for ophthalmic compositions. For example, sodium chloride, potassium chloride, magnesium chloride, calcium chloride, dextrose and/or mannitol are added to the composition to approximate physiological tonicity. Such an amount of tonicity agent will vary, depending on the particular agent to be added. In general, however, the compositions have a tonicity agent in an amount sufficient to cause the final composition to have an ophthalmically acceptable osmolality (generally about 150-450 mOsm, preferably 250-350 mOsm).

The pharmaceutically acceptable ophthalmic carrier used with the formulations of the invention may be of a wide range of types known to those of skill in the art. For example, the formulations of the invention are optionally provided as an ophthalmic solution or suspension, in which case the carrier is at least partially aqueous. Optionally, the formulations are ointments, in which case the pharmaceutically acceptable carrier comprises an ointment base. Preferred ointment bases herein have a melting or softening point close to body temperature, and any ointment bases commonly used in ophthalmic preparations are advantageously employed. Common ointment bases include petrolatum and mixtures of petrolatum and mineral oil.

The formulations of the invention are optionally prepared as a hydrogel, dispersion, or colloidal suspension. Hydrogels are formed by incorporation of a swellable, gel-forming polymer such as those set forth above as suitable thickening agents (i.e., MC, HEC, HPC, HPMC, NaCMC, PVA, or hyaluronic acid or a salt thereof, e.g., sodium hyaluronate), except that a formulation referred to in the art as a “hydrogel” typically has a higher viscosity than a formulation referred to as a “thickened” solution or suspension. In contrast to such preformed hydrogels, a formulation may also be prepared so as to form a hydrogel in situ following application to the eye. Such gels are liquid at room temperature but gel at higher temperatures (and thus are termed “thermoreversible” hydrogels), such as when placed in contact with body fluids. Biocompatible polymers that impart this property include acrylic acid polymers and copolymers, N-isopropylacrylamide derivatives, and ABA block copolymers of ethylene oxide and propylene oxide (conventionally referred to as “poloxamers” and available under the Pluronic® tradename from BASF-Wyandotte). The formulations can also be prepared in the form of a dispersion or colloidal suspension. Preferred dispersions are liposomal, in which case the formulation is enclosed within “liposomes,” microscopic vesicles composed of alternating aqueous compartments and lipid bilayers. Colloidal suspensions are generally formed from microparticles, i.e., from microspheres, nanospheres, microcapsules, or nanocapsules, wherein microspheres and nanospheres are generally monolithic particles of a polymer matrix in which the formulation is trapped, adsorbed, or otherwise contained, while with microcapsules and nanocapsules, the formulation is actually encapsulated. The upper limit for the size for these microparticles is about 5 um to about 10 um.

The formulations are optionally incorporated into a sterile ocular insert that provides for controlled release of the formulation over an extended time period, generally in the range of about 12 hours to 60 days, and possibly up to 12 months or more, following implantation of the insert into the conjunctiva, sclera, or pars plana, or into the anterior segment or posterior segment of the eye. One type of ocular insert is an implant in the form of a monolithic polymer matrix that gradually releases the formulation to the eye through diffusion and/or matrix degradation. With such an insert, it is preferred that the polymer be completely soluble and or biodegradable (i.e., physically or enzymatically eroded in the eye) so that removal of the insert is unnecessary. These types of inserts are well known in the art, and are typically composed of a water-swellable, gel-forming polymer such as collagen, polyvinyl alcohol, or a cellulosic polymer. Another type of insert that is used to deliver the present formulation is a diffusional implant in which the formulation is contained in a central reservoir enclosed within a permeable polymer membrane that allows for gradual diffusion of the formulation out of the implant. Optionally, osmotic inserts are used, i.e., implants in which the formulation is released as a result of an increase in osmotic pressure within the implant following application to the eye and subsequent absorption of lachrymal fluid.

The invention also pertains to ocular inserts for the controlled release of combinations of the metal complexer and transport enhancer. These ocular inserts are implanted into any region of the eye, including the sclera and the anterior and posterior segments. One such insert is composed of a controlled release implant containing a formulation that consists essentially of the active agent and a pharmaceutically acceptable carrier. The insert is a gradually but completely soluble implant, such as may be made by incorporating swellable, hydrogel-forming polymers into an aqueous liquid formulation. Alternatively, the insert is insoluble, in which case the agent is released from an internal reservoir through an outer membrane via diffusion or osmosis.

The term “controlled release” refers to an agent-containing formulation or fraction thereof in which release of the agent is not immediate, i.e., with a “controlled release” formulation, administration does not result in immediate release of the agent into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein refers to “sustained release” rather than to “delayed release” formulations. The term “sustained release” (synonymous with “extended release”) is used in its conventional sense to refer to a formulation that provides for gradual release of an agent over an extended period of time.

In one aspect, an ophthalmic formulation of the invention is administered topically. Optionally, topical ophthalmic products are packaged in multidose form. Preservatives may thus be required to prevent microbial contamination during use. Suitable preservatives include: chlorobutanol, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives. However, the ophthalmic compositions of the present invention are preferably preservative free and packaged in unit dose form.

The preferred compositions of the present invention are intended for administration to a mammal in need thereof, in particular to a human patient. In general, the doses used for the above described purposes will vary, but will be in an effective amount to eliminate or improve dry eye conditions. Generally, 1-2 drops of such compositions will be administered one or more times per day. For example, the composition can be administered 2 to 3 times a day or as directed by an eye care provider.

The results described herein demonstrate: (1) transient elevation of IOP, optic nerve trauma, and ischemia of ocular tissues induce T cell infiltration into the retina and T cell-mediated autoimmune attacks to RGCs and their axons; (2) small molecular weight hsps are pathogenic auto-antigens involved in these immune responses; (3) adoptive transfer of CD4+ T cells facilitated the second phase of glaucomatous RGC damage, while genetic ablation of T cell functions prevented RGC and axon degeneration induced after the elevated IOP returned to a normal range. Thus, the results described herein demonstrate a functional link between T cell-mediated autoimmune responses specific to small hsps and the development/prognosis of optic neuropathy in glaucoma, AION, and optic nerve trauma (e.g., an eye injury (e.g., blast injury that severs the optic nerve) or an ophthalmic tumor (e.g., a tumor on the optic nerve). Such conditions are identified using known methods, e.g., ischemia of the ocular tissues is identified by patient presentation with blurred vision and/or with an ophthalmic scope, and other methods described above.

### Mouse Model of Glaucoma

Prior to the invention described herein, there was not a suitable mouse model of glaucoma which allowed the utilization of genetic tools. Therefore, an inducible and reversible mouse model of elevated IOP was developed by injecting polystyrene microbeads to the anterior chamber without causing apparent inflammation or permanent damage to ocular structures. This mouse model enabled recapitulation of clinical conditions in glaucoma patients of whom IOP is elevated but then is controlled under a normal range due to drug treatment or a nature course of the disease. It allows identification of subsequent events evoked by the initial IOP elevation. Using this model, a functional link between the seemingly disparate processes—elevated IOP and induction of adaptive immune response was discovered. Elevation of IOP triggers T and B cell-mediated immune responses that continuously attack RGCs and axons and critically contribute to the progressive glaucomatous neurodegeneration. Blockade of the adaptive immune responses using a genetic approach abolishes optic neuropathy secondary to IOP elevation.

Demonstration of such a link established a novel pathogenic mechanism underlying RGC and optic nerve damage in glaucoma, and implicates an involvement of adaptive immune mechanisms in the pathogenesis of other neurodegenerative processes. Treatments currently available for CNS autoimmune disorders, such as Multiple Sclerosis (e.g., corticosteroids, plasma exchange (plasmapheresis), beta interferons, glatiramer (copaxone), fingolimod (gilenya), natalizumab (tysabri), and mitoxantrone (novantrone), are applicable to glaucoma.

### Elevated Intraocular Pressure and Heat Shock Proteins

Glaucoma is characterized by progressive damage to RGCs and their axons, leading to permanent vision loss. It is the most widely spread neurodegenerative disorder, affecting 70 million people worldwide (Quigley, H. A. & Broman, A. T. Br J Ophthalmol 90, 262-267 (2006)). POAG is the most common form of glaucoma. Typically, POAG is associated with raised intraocular pressure, but glaucomatous neuronal damage also occurs in individuals who exhibit a normal range of IOP (Flammer, J. & Mozaffarieh, M. Surv Ophthalmol 52 Suppl 2, S162-173 (2007), suggesting the presence of secondary events. Consistent with this notion, treatments that are directed at lowering IOP often do not completely stop the progression of vision loss. Glaucoma patients whose IOP appears to be perfectly controlled continue to manifest neuronal loss and visual field deterioration (McKinnon et al., Am J Manag Care 14, S20-27 (2008); Walland et al., Clin Experiment Ophthalmol 34, 827-836 (2006)). Elevation of IOP triggers a sequence of events that may lead to secondary damage to the optic nerve and RGCs, in part, by inducing stress responses and expression of stress proteins, such as heat shock proteins (hsps; Tezel et al., Arch Ophthalmol 118, 511-518 (2000); Park et al., Investigative ophthalmology & visual science 42, 1522-1530 (2001)).

Hsps are a class of functionally related, highly conserved proteins involved in the folding and unfolding of other proteins. Many hsps are highly immunogenic, and their expression is increased when cells are exposed to elevated temperatures or other stress. The dramatic upregulation of the heat shock proteins is a key part of the heat shock response, and is induced primarily by heat shock factor (HSF). Hsps are named according to their molecular weight.

The nucleic acid sequence of human hsp27 is provided in GenBank Accession Number X54079.1 (GI:32477), incorporated herein by reference. The amino acid sequence of human hsp27 is provided in GenBank Accession Number BAB17232.1 (GI:11036357), incorporated herein by reference. The nucleic acid sequence of hsp60 is provided in GenBank Accession Number M34664.1 (GI:184411), incorporated herein by reference. The amino acid sequence of hsp60 is provided in GenBank Accession Number AAF66640.1 (GI:7672784), incorporated herein by reference.

Enhanced expression of hsps under stress can unveil previously hidden antigenic determinants to initiate and perpetuate autoimmune responses (Rajaiah, R. & Moudgil, K. D. Autoimmun Rev 8, 388-393 (2009)). Heat shock proteins participate in the induction and propagation of several autoimmune diseases, including rheumatoid arthritis, atherosclerosis and type I diabetes (Young, D. B. Current opinion in immunology 4, 396-400 (1992); Wick et al., Annu Rev Immunol 22, 361-403 (2004); van Eden et al., Nat Rev Immunol 5, 318-330 (2005)). Emerging evidence suggests that the etiopathogenesis of glaucomatous neuronal damage may also involve autoimmune responses associated with hsps (Wax, M. B. & Tezel, G. Experimental eye research 88, 825-830 (2009); Tezel, G. & Wax, M. B. Curr Opin Ophthalmol 15, 80-84. (2004)). Glaucoma patients have elevated levels of autoantibodies to hsps and retinal antigens and abnormal subpopulation of T cells (Tezel, G. & Wax, M. B. Curr Opin Ophthalmol 15, 80-84. (2004); Wax, M. B., Yang, J. & Tezel, G. J Glaucoma 10, S22-24 (2001); Grus et al., J Glaucoma 17, 79-84 (2008)). Immunization of rats with hsp27 and hsp60 induced optic neuropathy that simulated glaucomatous RGC and axon damage in human patients (Wax et al., J Neurosci 28, 12085-12096 (2008)). However, there is also evidence that supports a neuroprotective role of autoreactive immune cells in glaucoma. For instance, myelin-specific T cells protected neurons from secondary degeneration in an experimental model of glaucoma (Schori et al., Proc Natl Acad Sci USA 98, 3398-3403 (2001)).

Prior to the invention described herein, a central unresolved question was whether induction of autoimmune responses is a critical mechanism by which IOP elevation leads to the development of glaucomatous neurodegeneration. Despite the correlative evidence from both clinical and experimental studies, prior to the invention described herein, unequivocal evidence supporting a direct role of autoimmune responses in neuronal damage in glaucoma was lacking.

Prior to the invention described herein, the study of pathogenesis of glaucoma was hampered by the lack of a suitable mouse model that allows genetic dissection of cellular and molecular pathways involved in glaucoma pathogenesis. As described above, a mouse model of ocular hypertension was developed by injecting polystyrene microbeads into the anterior chamber without causing apparent inflammation or permanent damage to ocular structures (Chen et al., Investigative ophthalmology & visual science 52, 36-44 (2011); Sappington et al., Investigative ophthalmology & visual science 51, 207-216 (2010)). This mouse model recapitulates key clinical features of POAG, and allows identification of subsequent events induced by the initial IOP elevation.

Described herein is a functional link between the seemingly disparate processes—elevated IOP and induction of autoimmune responses in pathogenesis of glaucoma. As described in detail below, ocular hypertension induced elevated expression of hsp27 in RGCs, and triggered CD4 T cell responses that are required and sufficient for progressive glaucomatous neurodegeneration. Additionally, patients with POAG were also characterized by a significantly increased level of hsp27 reactive T cells as compared to age-matched healthy individuals. These findings described herein establish CD4+ T cell-mediated autoimmune responses to hsp27 as a major pathogenic mechanism underlying progressive RGC and optic nerve degeneration in glaucoma. The results described in detail below explain the ineffectiveness of treatment strategies that are directed solely to lowering IOP, and provide unique approaches to prevent or inhibit vision loss in glaucoma.

### Example 1

### Autoimmune CD4 T Cell Responses to Heat Shock Protein 27 Mediate Progressive Neurodegeneration in Glaucoma

Glaucoma is a neurodegenerative disease and leading cause of irreversible blindness. Although elevated IOP is known as a major risk factor, prior to the invention described herein, the underlying cellular and molecular mechanisms through which an elevation of IOP leads to neuronal damage were unknown. As described in detail below, elevated IOP induced a progressive (secondary) neurodegeneration by stimulating autoreactive CD4+ T cell responses to hsp27. As described herein, while glaucomatous neurodegeneration was readily induced by elevation of IOP in wild-type mice, the secondary neuronal damage was abolished in the absence of T cells. Additionally, transfer of T cells from wild-type mice with glaucoma restored the secondary neuronal and axon degeneration in T cell-deficient mice. As described in detail below, elevated IOP stimulated hsp27 expression in the retina and CD4+ T cell responses, and transfer of hsp27-specific CD4+ T cells exacerbated neurodegeneration in wild-type mice. In addition, patients with primary open-angle glaucoma exhibited a 6-fold increase in hsp27-responsive T cells in the peripheral blood as compared to normal individuals. The findings presented herein demonstrate a critical role of CD4+ T cell-mediated autoimmune responses to hsp27 in the pathogenesis of POAG. Thus, described herein are methods for preventing and limiting vision loss in glaucoma.

### Mice

C57BL/6J (B6) mice were purchased from Charles River Breeding Laboratories. Rag1−/−, TCR−/−, Igh6−/− mice, all on the B6 background, and DBA/2J mice were purchased from the Jackson Laboratories.

### Induction of IOP Elevation in Mice

Induction of IOP in mice was described previously (Chen et al., Investigative ophthalmology & visual science 52, 36-44 (2011)). Briefly, mice were anesthetized supplemented by topical proparacaine HCl (0.5%; Baush & Lomb Incorporated, Tampa, Fla.). Elevation of TOP was induced unilaterally in adult mice by anterior chamber injection of polystyrene microbeads with a uniformed diameter of 15 μm (Invitrogen), which had been re-suspended in PBS at a final concentration of 5.0×10⁶ beads/ml. The control group received an injection of 2 μl PBS to the anterior chamber. In all experimental groups, TOP was measured every other day in both eyes using a TonoLab tonometer (Colonial Medical Supply) and performed as previously described (Saeki et al., Current eye research 33, 247-252 (2008)).

### Quantification of Axon and RGC Loss

A standard procedure for quantification of RGC axon loss in optic nerve sections was used, e.g., as described in Cho et al., J Cell Sci 118, 863-872. (2005). Axonal density was calculated, and the percentage of axon loss was determined by comparing with the axon density calculated from corresponding regions of the contralateral control eyes. RGC loss was assessed quantitatively in retinal flat-mounts that were incubated with a primary antibody against a RGC specific marker, β-III-tubulin (Fournier, A. E. & McKerracher, L. Biochem Cell Biol 73, 659-664 (1995); Fitzgerald et al., Investigative ophthalmology & visual science (2009); Tuj1; Sigma-Aldrich, St.), followed by a Alexa Fluor 488-conjugated secondary antibody. The degree of RGC loss was assessed as previously described (Chen et al., Investigative ophthalmology & visual science 52, 36-44 (2011)). The total numbers and densities of RGCs were calculated, and the percentage of RGC loss was determined by comparing RGC number with that obtained from the corresponding regions of the contralateral control eyes.

### Isolation and Adoptive Transfer of CD4+ T Cells

Spleens were mechanically homogenized, and cells were suspended in RPMI media (Sigma) containing 10% FBS, 1% penstrep. and 1% L-glutamine, and red blood cells (RBCs) were lysed with RBC lysis buffer (Sigma). CD4+ T cells were purified using an auto magnetic-activated cell sorting (MACS) Separator and a CD4+ T Cell Isolation Kit (Miltenyi Biotec) according to the manufacturer's protocol. Briefly, CD4+ T cells were negatively selected from splenocytes of hsp27-immunized mice or mice with high IOP by depletion with a mixture of lineage-specific biotin conjugated antibodies against CD8 (Ly-2), CD11b (Mac-1), CD45R (B220), CD49b (DX5), Ter-119, and antibiotin microbeads. The procedure yielded an over 90% purity of CD4+ T cells as assessed by flow cytometry. CD4+ T cells (2×10⁸ cells) suspended in 200 μl PBS) were adoptively transferred into recipient mouse via tail vein injection. Control group received same numbers of CD4+ T cells isolated from mice with normal IOP or from ovalbumin (Ova) immunized mice.

### ELISPOT Assays

Mouse interferon gamma (IFN-γ) enzyme-linked immunosorbent spot (ELISPOT) assay (eBioscience) was used to determine frequencies of IFN-γ-producing T cells in response to hsp27 or hsp60 (Sigma Aldrich). ELISPOT plates (Multiscreen-MAIPS4510) pre-coated with 100 μl/well of capture antibody were blocked with 200 μl/well of complete RPMI-1640. Purified CD4+ T cells (2×10⁶ cell/ml) were added and incubated with antigens, including hsp27, hsp60, IRBP, and MBP (invitrogen) at a final concentration of 10 μg/ml for 48 hours. Cell cultures incubated alone or with Ova were used as controls. Results are shown as mean antigen-specific spot forming cells (SFC) after background subtraction from control wells containing no antigen.

### Enzyme-Linked Immunosorbent Assay (ELISA)

Mice injected with microbead or phosphate buffered saline (PBS; controls) were sacrificed. Peripheral blood and the serum were collected. Ninety-six-well plate (Nunc) was pre-coated with recombinant human hsp27 protein (1 μg/ml) or hsp60 followed by incubation with 10% normal goat serum before the diluted serum samples (1:10), and anti-hsp27 antibody (positive control) were added and incubated for 2 hours at room temperature. Serum IgG levels were detected by incubation with HRP-conjugated anti-mouse IgG for 45 min at room temperature. Serum levels of hsp27 autoantibody was detected by incubating the serum samples with TMP substrate (Sigma), and then measured at excitation wavelength 405 nm using XFlour4 software. Each sample was performed in triplicate.

### Collection and Preparation of Human Blood Samples and T Cell Assays

Patients at 40-60 years old who had been diagnosed with POAG with unambiguous clinical evidence of pathological “cupping” of the optic nerve head and documentation of visual field loss on visual field testing were recruited for this study. Patients who also had history of any other retinal diseases (e.g., diabetic retinopathy, retinal detachment, macular degeneration) or neurological conditions had been excluded from this study. Control subjects recruited did not show any evidence of optic nerve or CNS damage from any cause and ultimately did not have any significant visual or neurological disorder. Sixteen ml of venous blood was drawn from each volunteer into a vacutainer CPT tube (Becton Dickinson) with sodium citrate and processed according to the manufacturer's instructions. The peripheral blood mononuclear cells (PBMCs) were resuspended at a concentration of 1.0×10⁷ cells/ml in RPMI with 20% heat-inactivated fetal bovine serum plus 10% dimethyl sulfoxide. ELISPOT and ELISA assays were performed as described above.

### Delayed Type Hypersensitivity Assay (DTH)

Thickness of the mouse ear was measured using a micrometer before antigen stimulation. Dorsal side of the mouse ears were injected with 10 μl human recombinant hsp27 (1 μg/μl; Enzo Life Science), hsp60, or a control antigen, MBP or IRBP (1 μg/μl; Invitrogen). The ear thickness of the injected ear was measured again after 24 hr, and change of ear thickness was calculated.

### Hsp27 Immunization

To immunize mice, 50 μl human recombinant hsp27 (50 μg; Enzo Life Science) was emulcified with 50 μl CFA emulsion and injected subcutaneously to adult B6 mice. Two to 3 weeks late, immune responses to hsp27 was analyzed by DTH and ELISPOT assays.

### Transient Elevation of IOP Induces Progressive RGC and Axon Degeneration

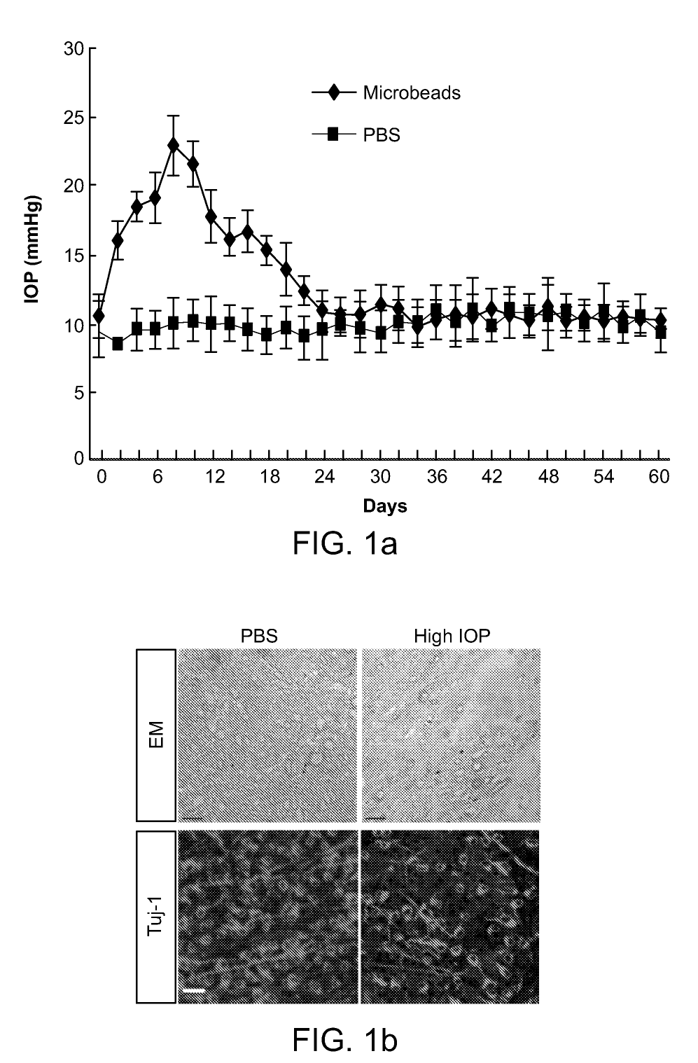

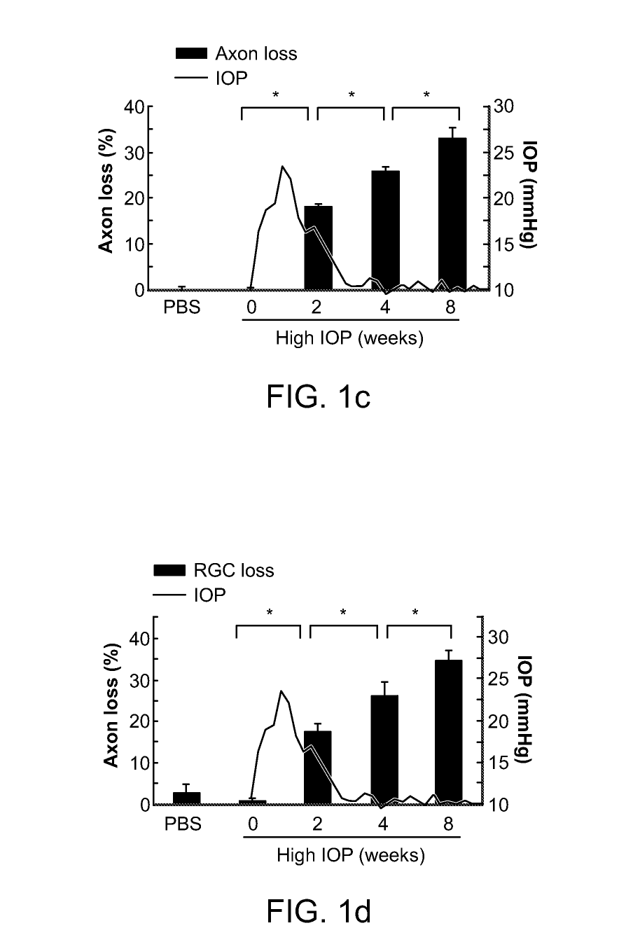

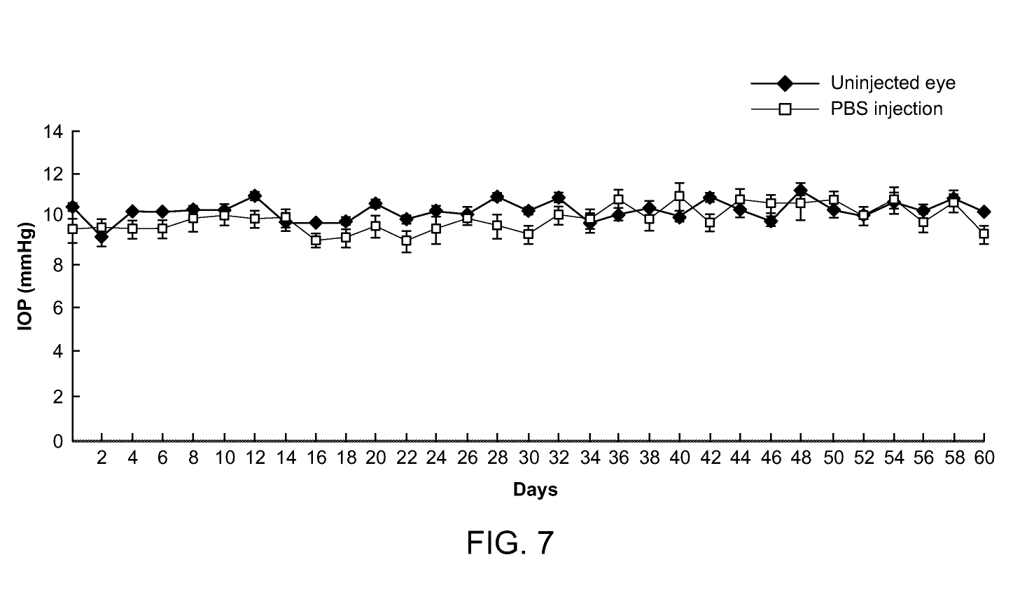

IOP reflects a balance between the rates of aqueous humorous that flows into and out of the eye. To investigate how an elevated IOP leads to glaucoma neurodegeneration, 15 μm polystyrene microbeads were injected into the anterior chamber of adult C57BL/6 (B6) mice and measured IOP every two days for 60 days (Chen et al., Investigative ophthalmology & visual science 52, 36-44 (2011)). A single injection blocked the aqueous outflow, and resulted in a significant elevation of IOP that lasted for approximately 3 weeks with the peak elevation around 8 days post injection (FIG. 1A). In contrast, the contralateral eyes with PBS injection or no injection did not show significant change in IOP value (FIG. 1A and FIG. 7).

The 3-week transient elevation of IOP induced a progressive neurodegeneration that extends far beyond the period of IOP elevation in these mice. The number of axons in the optic nerves and RGCs in the retinas was quantified by immunofluorescent staining and electron microscopy at 2, 4 and 8 weeks post injection (FIG. 1B). Significant loss of axons and RGCs was detected as early as 2 weeks post injection when IOP was still elevated (FIG. 1C-D). Importantly, the loss continued from 2 to 4 and 4 to 8 weeks post injection when the IOP had returned to the normal range. These data indicate that elevation of IOP triggers a subsequent event that critically contributes to the progressive RGC and optic nerve damage secondary to the IOP elevation.

Glaucomatous Neurodegeneration is Associated with T Cell Infiltration and Complement Deposition in the Retina

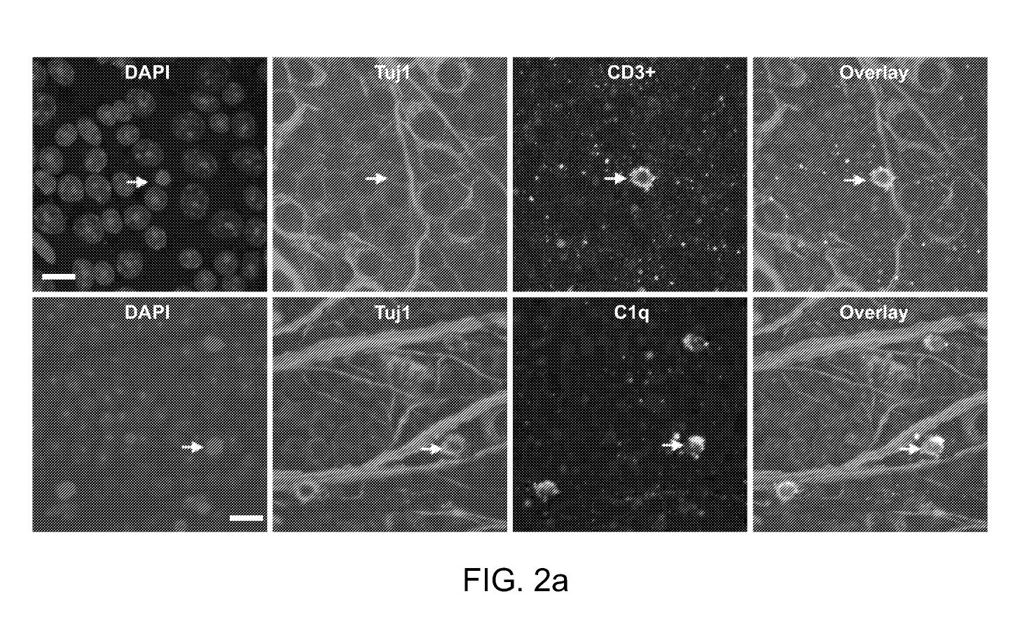

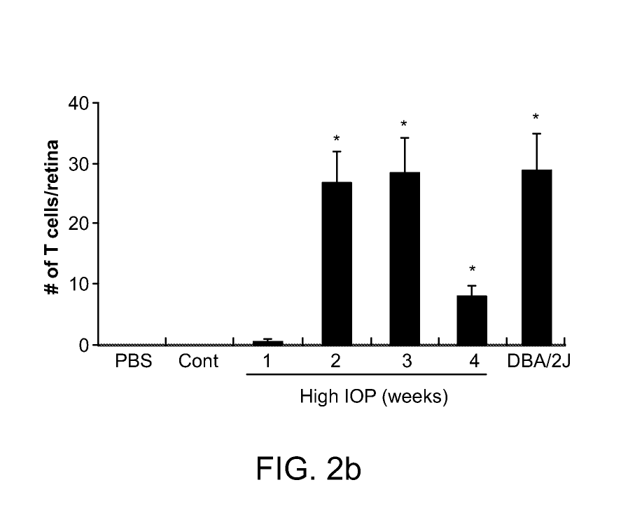

To investigate whether the immune system is involved in the glaucomatous neuronal damage, T cell infiltration and complement deposition was examined in the retinas, and serum IgG levels were examined in the microbead-injected mice. Immunofluorescent staining with an anti-CD3 antibody detected infiltration of T cells in the retinas of microbead-injected eyes, but not the PBS-injected or uninjected eyes (FIG. 2A and FIG. 2B). T cell infiltration was detected 2 and 3 weeks post microbead injection, but was significantly reduced by 4 weeks post injection when IOP has returned to the normal range (FIG. 2B). Similarly, deposition of C1q, a marker of the classical complement cascade that is activated by antigen-antibody complexes, was detected in the ganglion cell layer (GCL) of the eyes with ocular hypertension, but not in the control eyes (FIG. 2A). Double immunolabeling of CD3 or C1q with a RGC specific marker Tuj1 showed that both infiltrated T cells and C1q deposition were closely associated with RGCs and their nerve fibers (FIG. 2A). In addition, by 2-8 weeks post injection, a ˜3-fold increase in the serum IgG level was observed in mice that were injected with microbeads as compared to uninjected or PBS injected mice (FIG. 2D).

To preclude the possibility that retinal T cell infiltration and increased titers of serum IgGs are associated with microbead injection, DBA/2J mice, a well-defined mouse model of an inherited form of glaucoma were analyzed. DBA/2J mice develop ocular hypertension and neuronal damage at about 6-8 months of age (Chang et al., Nat Genet 21, 405-409 (1999); Anderson et al., Nat Genet 30, 81-85 (2002)). Consistent with the observations in the microbead-induced ocular hypertension in B6 mice, T cell infiltration and an elevated serum IgG level were detected in 8 month-old (FIG. 2B and FIG. 2C), but not in 2 month-old DBA/2J mice. Together, these results demonstrate that glaucomatous neurodegeneration initiated by the elevated TOP is associated with immune attacks in the retinas.

### T Cell Deficiency Attenuates the Secondary Glaucomatous Neurodegeneration

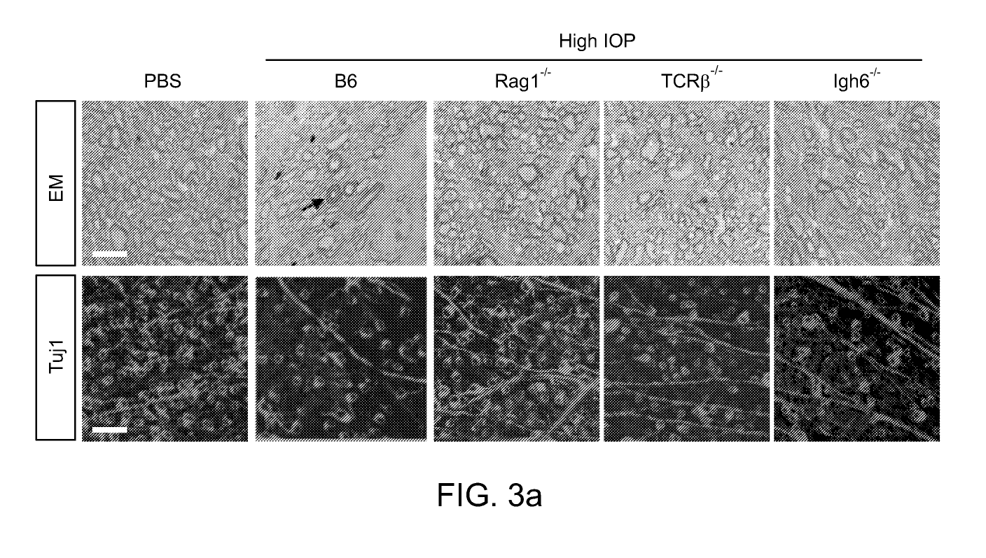

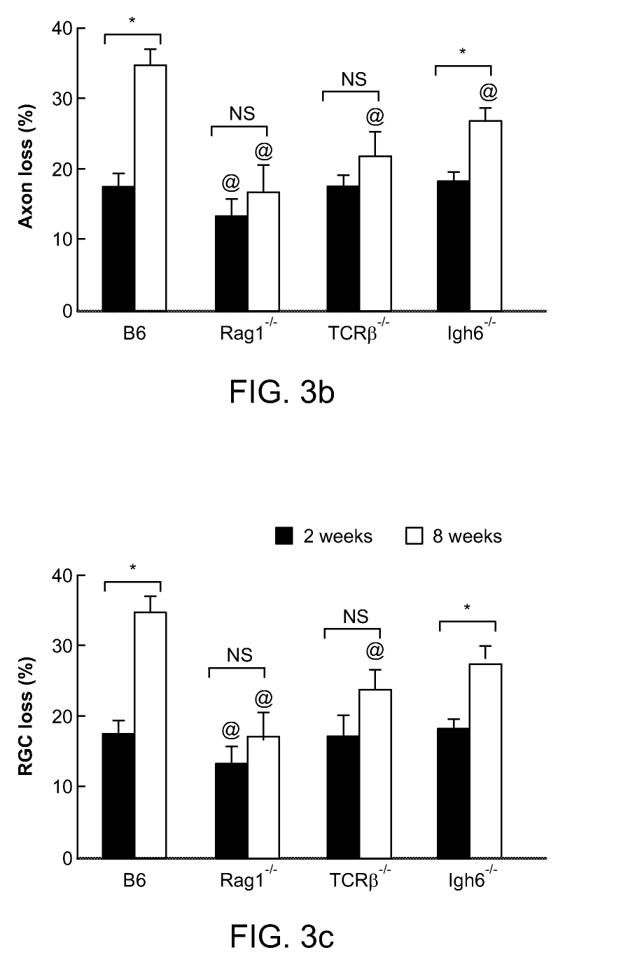

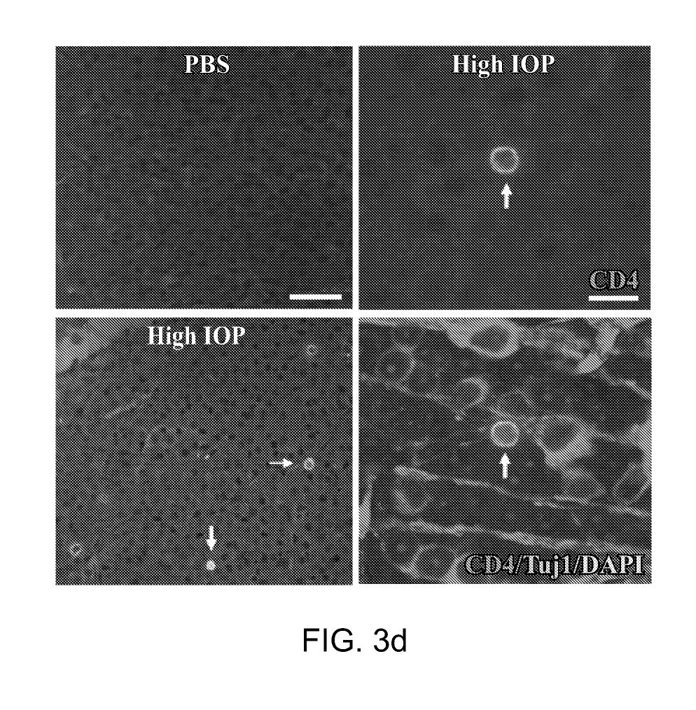

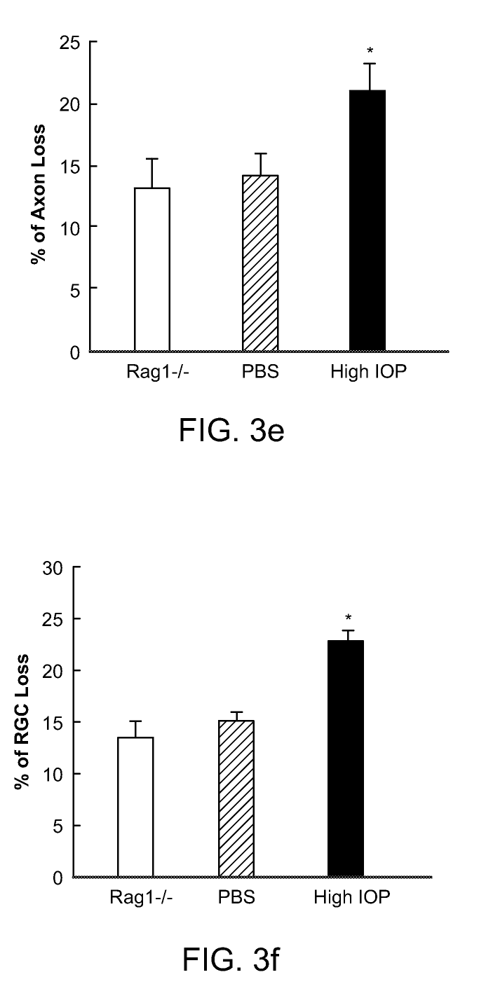

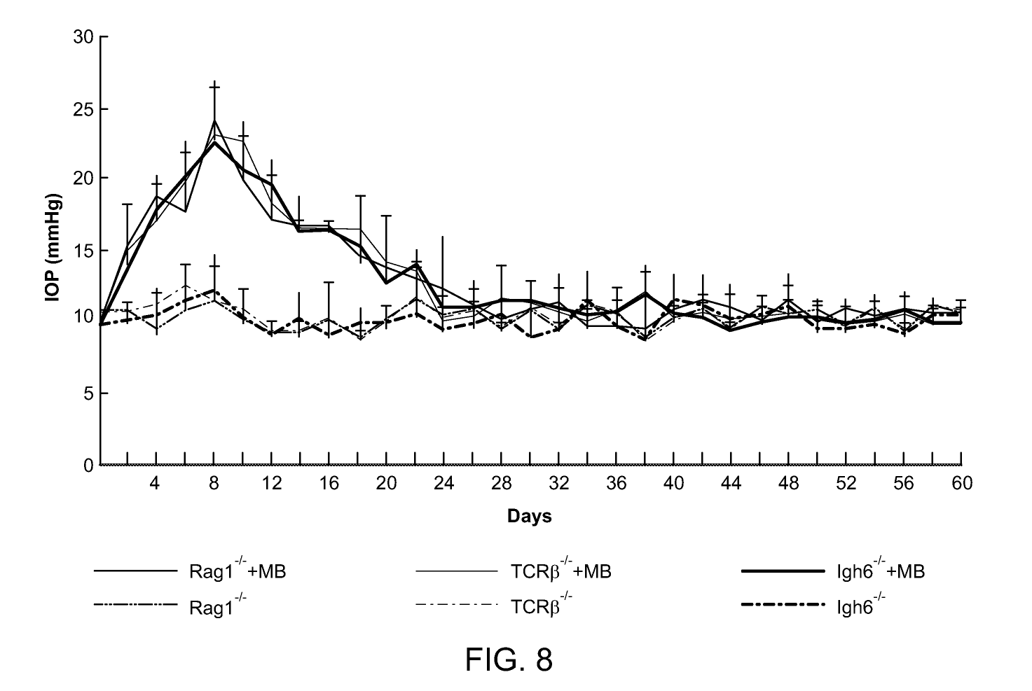

To determine the role of immune responses in glaucomatous neural damage, the disease development in Rag1−/− mice that are deficient in both T and B cells was examined (Mombaerts et al., Cell 68, 869-877 (1992)). Injection of microbeads to the anterior chamber of Rag1−/− mice induced TOP elevation that had the same kinetics as in the wild-type B6 mice (FIG. 8). However, unlike in the wild-type mice, transient elevation of TOP did not induce T cell infiltration or C1q deposition in the retina of Rag1−/− mice. Correspondingly, the loss of axons and RGCs in the microbead-injected eyes was significantly diminished in Rag1−/− mice at both 2 and 8 weeks post injection as compared to that in the wild-type mice at the corresponding time points (FIG. 3B and FIG. 3C). Notably, there was no significant increase in the loss of axons and RGCs in Rag1−/− mice between 2 and 8 weeks post injection. These results demonstrate that the axon and RGC loss at 2 weeks post microbead injection in Rag1−/− mice is caused primarily by the elevated TOP whereas the further axon and RGC loss between 2 and 8 weeks likely results from immune attacks. Deficiency in T and B cells significantly attenuates the secondary glaucomatous neurodegeneration.

To distinguish the contribution of T cell- or B cell-mediated responses to the secondary neuronal damage, axon and RGC loss was compared in T cell- (TCRβ−/−) (Mombaerts et al., Nature 360, 225-231 (1992)) and B cell-deficient (Igh6−/−) mice. Anterior chamber injection of microbeads induced elevation of TOP in TCRβ−/− and Igh6−/− mice with the same kinetics as that in the wild-type B6 mice (FIG. 8). The loss of axons and RGCs in the optic nerve and retinas were similar in wild-type, TCRβ−/−, and Igh6−/− mice 2 weeks post microbead injection (FIG. 3A-FIG. 3C), indicating deficiency of T or B cells does not attenuate the primary neurodegeneration induced directly by the elevated IOP. As in Rag1−/− mice, T cell deficiency (TCRβ−/−) significantly inhibited the secondary degeneration of axons and RGCs from 2 to 8 weeks post microbead injection. In contrast, in Igh6−/− mice, a marked loss of RGCs and axons still occurred from 2 to 8 weeks post microbead injection. There was only a moderate reduction of axon and RGC loss in Igh6−/− mice as compared to that of wild-type mice at week 8. Together, these results demonstrate an essential requirement of adaptive immunity, particularly T cell-mediated responses, in the IOP-initiated glaucoma by activating a secondary mechanism of RGC and axon degeneration.

Adoptively Transferred T Cells from High IOP Mice Restore the Secondary Neurodegeneration in Rag1−/− Mice

To test whether T cells are sufficient to induce the secondary glaucomatous neural damage, T cells from high IOP mice were transferred into Rag1−/− mice and analyzed the disease development in the recipient mice. Because of a critical role of CD4+ T cells in autoimmune diseases (Goverman, J. Nat Rev Immunol 9, 393-407 (2009)), CD4+ T cells were focused on. Splenic CD4+ T cells were isolated from B6 mice at 2 weeks post microbead injection or from PBS-injected control mice and injected via tailvein into recipient Rag1−/− mice (1.0×10⁸ cells per recipient) that had also been induced to develop high IOP for 2 weeks by a single injection of microbeads to the anterior chamber. Two weeks post cell transfer (or 4 weeks after initial microbead injection), recipient mice were sacrificed and analyzed for axon and RGC levels in retinas. Rag1−/− mice without T cell transfer or transferred with CD4+ T cells from PBS-treated B6 mice had similar numbers of axons and RGCs (FIG. 3D-FIG. 3F). In contrast, Rag1−/− mice transferred with CD4+ T cells from B6 mice with high IOP displayed a significant increase in axon and RGC loss (FIG. 3E-FIG. 3F). Consistently, T cell infiltration was detected in the retinas of Rag1−/− mice that were transferred with CD4+ T cells from high IOP mice, but not PBS injected control mice (FIG. 3D). Adoptive transfer of total IgGs from B6 mice with elevated IOP to Rag1−/− mice did not result in any significant loss of axons and RGCs. These results demonstrate that CD4+ T cells from diseased mice are sufficient to induce the secondary neurodegeneration in Rag1−/− mice.

### Hsp27 is an RGC Associated Autoantigen in the Elevated IOP-Initiated Autoimmune Responses

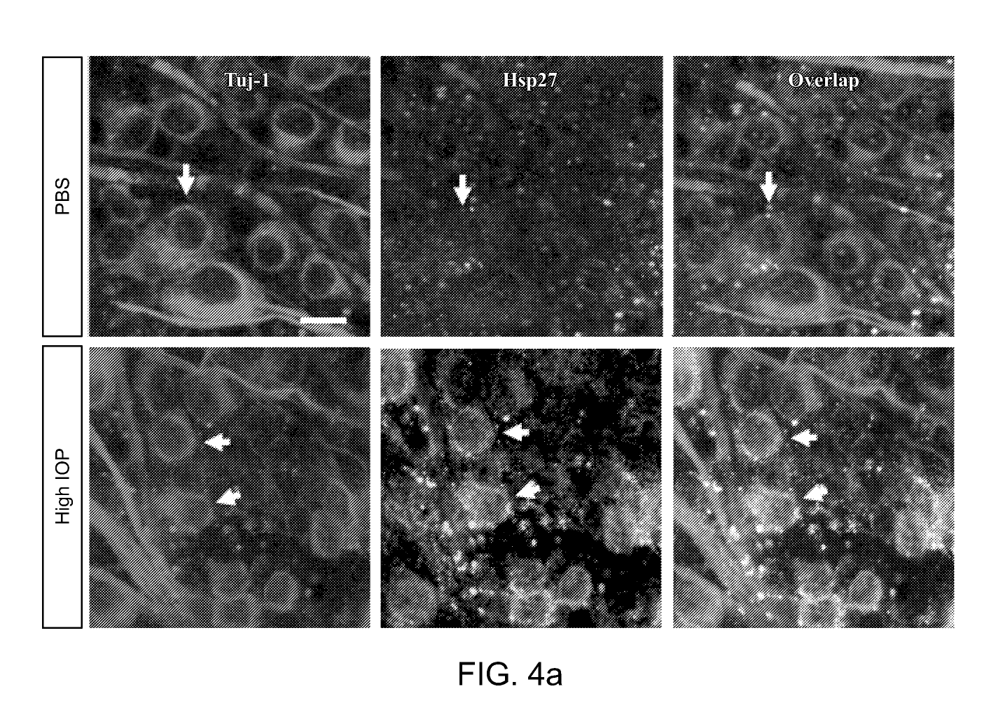

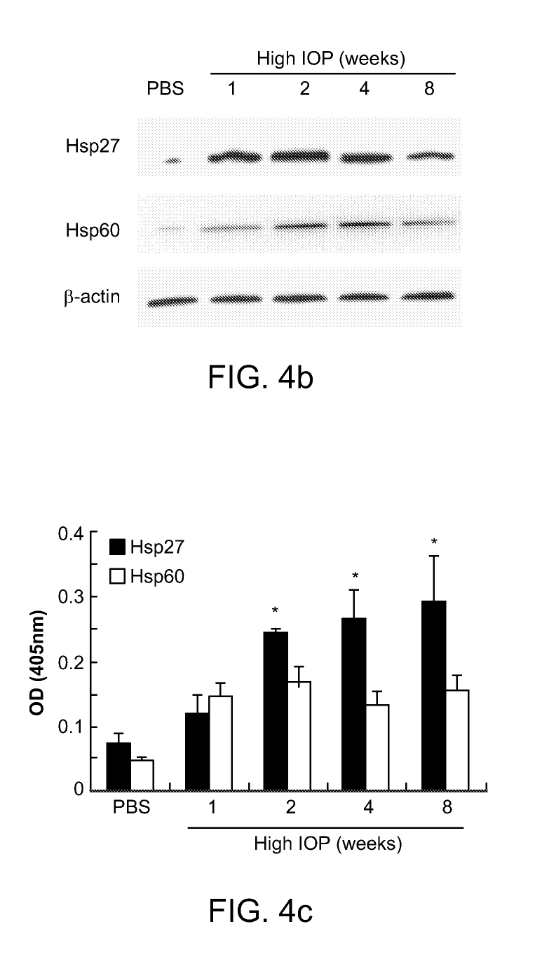

Next, antigens were investigated that might have stimulated CD4+ T cell response following elevation of IOP. Hsps were examined because autoimmune responses to them have been implicated in glaucomatous neural damage (Wax, M. B. & Tezel, G. Experimental eye research 88, 825-830 (2009)). Under the normal IOP, only a low level of hsp27 and hsp60 was detected in the mouse retina (FIG. 4A and FIG. 4B). Elevation of IOP resulted in upregulation of hsp27 expression in the retina within a week and lasted for over 8 weeks post microbead injection (FIG. 4A and FIG. 4B). Double-immunolabeling with Tuj1 antibody and anti-hsp27 showed that hsp27 was expressed by RGCs (FIG. 4A). hsp60 expression was also upregulated after IOP elevation, but to a lesser extent than hsp27 (FIG. 4B) and it did not co-localize with RGCs. Moreover, significant increase in serum levels of autoantibodies specific for hsp27 and hsp60 were detected in mice at 2, 4 and 8 weeks post-microbead injection (FIG. 4C).

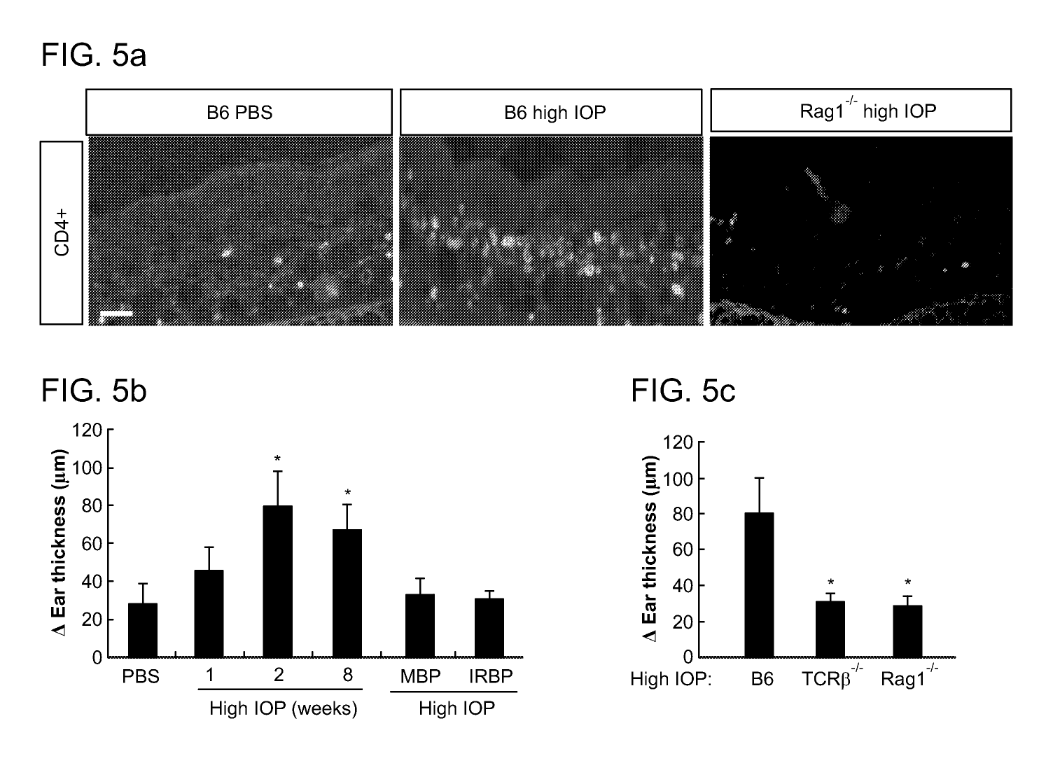

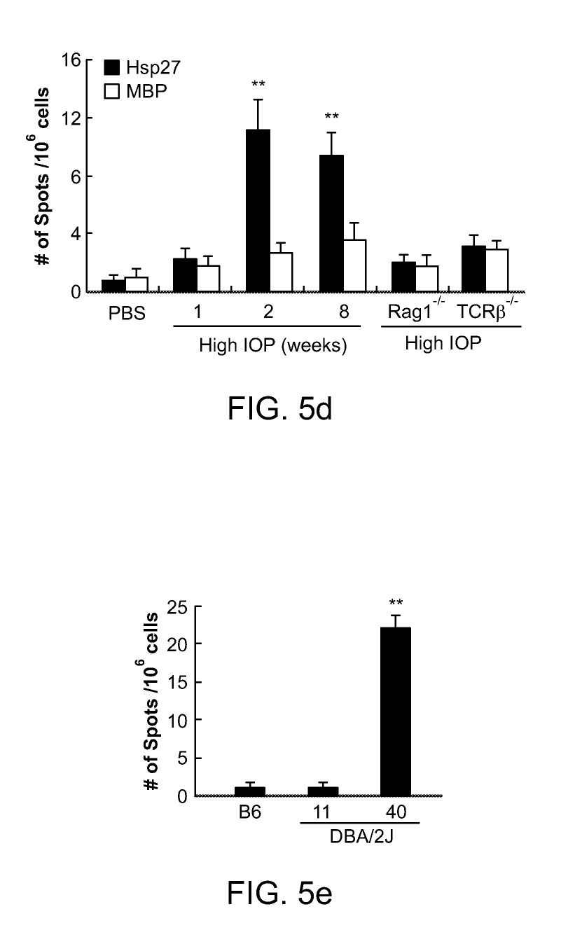

To investigate whether the elevated IOP induces CD4+ T cell response to hsp27, the induction kinetics of hsp27-specific T cell responses following injection of microbeads were determined by delayed type hypersensitivity (DTH) assay. B6 mice were injected with microbeads or PBS. One, 2 and 8 weeks later, mice were injected intradermally with recombinant hsp27 in the ears, and T cell infiltration and DTH response were measured. Much more abundant CD4+ T cells were detected in the ear of B6 mice with high IOP than control B6 mice with normal IOP 24 h after intradermal hsp27 injection (FIG. 5A). Coinciding with T cell infiltration into the retina, positive DTH responses (significantly increase in ear thickness) to hsp27 were detected in B6 mice with high IOP as early as 2 weeks post microbead injection and were still detected 8 weeks post microbead injection (FIG. 5B). Consistently, no significant increase in ear thicknesses was induced in ocular hypertensive Rag1−/− or TCRβ−/− mice, or in B6 mice with a normal IOP, or in B6 mice with high IOP, but challenged with irrelevant antigens, interphotoreceptor retinoid-binding protein (IRBP), or myelin basic protein (MBP) (FIG. 5B and FIG. 5C). Corroborating the DTH responses, significantly higher frequencies of IFN-γ secreting cells were induced by hsp27 in splenocytes from B6 mice 2 and 8 weeks post microbead injection as compared to PBS injected B6 mice or microbead injected Rag1−/− and TCRβ−/− mice (FIG. 5D). The induction of T cell response was hsp27 specific, as stimulation with MBP did not induce any increase in frequency of IFN-γ secreting T cells. In addition, a significantly increased frequency of IFN-γ secreting cells was induced by hsp27 in splenocytes of old (40 weeks) DBA/2J mice but not young (11 weeks) DBA/2J or old (40 weeks) B6 mice (FIG. 5E). Taken together, these results demonstrate that elevation of IOP stimulates hsp27 expression, which in turn leads to the induction of an autoreactive anti-hsp27 CD4+ T cell response.

### Hsp27 is a Pathogenic Autoantigen in Glaucomatous Neurodegeneration

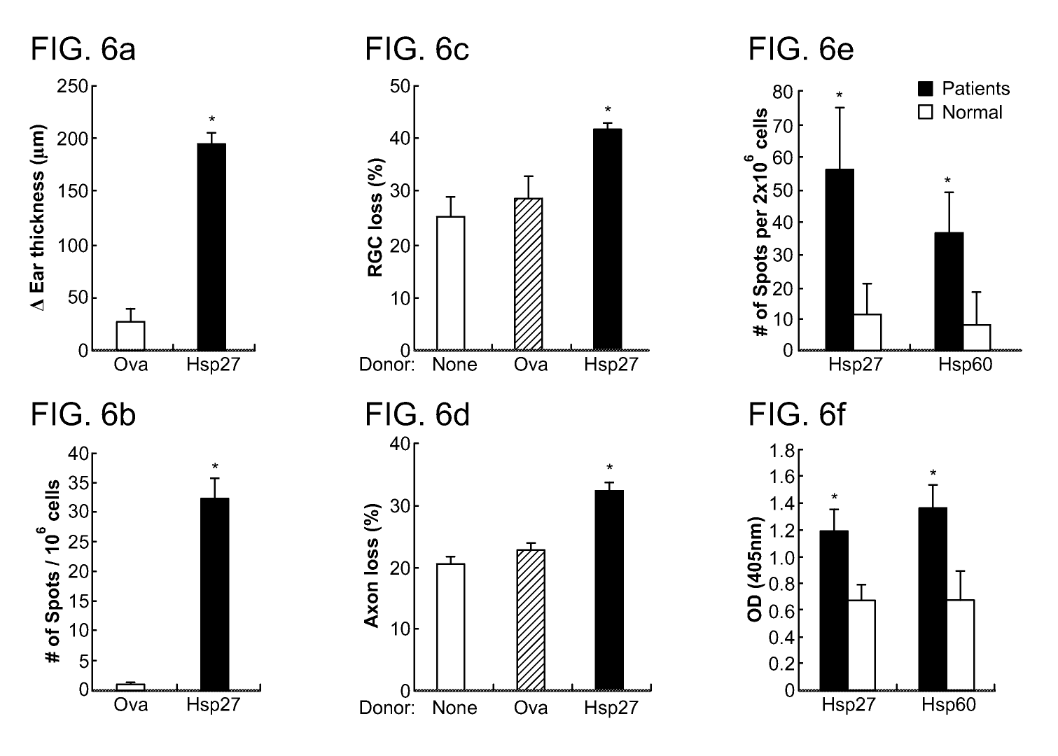

Next, the role of hsp27-specific CD4 T cells in glaucomatous neurodegeneration was determined by adoptive transfer. B6 mice were immunized with hsp27 or ovalbumin in IFA. Successful immunization of mice by hsp27 was confirmed by DTH and ELISPOT assays (FIG. 6A and FIG. 6B). Two weeks later, total CD4+ T cells, containing hsp27-specific cells, were isolated from the spleen and adoptively transferred into B6 mice that had been induced to develop high IOP for 2 weeks. Mice received CD4+ T cells from the hsp27-immunized mice displayed accelerated axon and RGC degeneration as compared to those received CD4+ T cells from Ova-immunized or control mice (FIG. 6C and FIG. 6D). Thus, hsp27-specific CD4+ T cells are capable of inducing secondary neuronal damage.

### Elevated Hsp27-Specific T Cells and Antibodies are Present in Glaucoma Patients

To investigate if induction of hsp27 specific T cell responses is associated with human glaucoma, the frequencies of hsp27 responsive T cells in the peripheral blood and hsp27-specific antibodies in the sera of POAG patients and age-matched healthy controls were analyzed. Eleven patients with POAG and 8 age-matched healthy individuals were enrolled. Remarkably, a 6-fold increase in frequency of hsp27 responsive T cells was detected in the patient's peripheral blood cells than in the age-matched healthy subjects (FIG. 6E). A 6-fold increase in frequency of hsp60-responsive T cells was also detected in patient compared to controls. In addition, a 2-fold increase in the titer of hsp27- or hsp60-specific autoantibodies was detected in the patient sera compared to control sera (FIG. 6F). These results demonstrate that elevated immune responses to hsp27 and hsp60 also occur in glaucoma patients.

### Involvement of Adaptive Immunity in the Etiology of Glaucoma

The results presented herein unequivocally demonstrate that autoreactive CD4+ T cells are required and sufficient to induce the progressive (secondary) degeneration of RGCs and axons initiated by elevated IOP. Elevated IOP induces a transient T cell infiltration into the retina. This transient infiltration of T cells into the affected parenchyma tissues during disease processes is also observed in hsp27 immunized model of optic neuropathy (Wax et al., J Neurosci 28, 12085-12096 (2008)) or other models of immune-mediated neuropathy, including experimental autoimmune encephalomyelitis or uveitis (Ludowyk et al., Journal of neuroimmunology 37, 237-250 (1992); Verhagen et al., Journal of neuroimmunology 53, 65-71 (1994); de Vos et al., Investigative ophthalmology & visual science 41, 3001-3010 (2000)). It implicates T cell involvement in initiating the immune responses leading to neurodegeneration. Importantly, T cell deficiency abolishes the secondary RGC and axon degeneration. Conversely, adoptive transfer of CD4+ T cells from diseased mice restores the secondary RGC and axon degeneration in T cell-deficient recipients. In contrast, B cell deficiency only has a modest effect on disease progression, and injection of total IgG antibodies from diseased mice does not have detectable effect. As described herein, hsp27 is a key pathogenic autoantigen because transfer of hsp27-specific CD4+ T cells exacerbates the disease severity initiated by IOP elevation. Furthermore, the relationship among IOP elevation, induction of hsp27 autoreactive CD4+ T cells, and secondary RGC and axon degeneration was explored. Elevation of IOP induced expression of hsps, which in turn stimulate CD4+ T cells responses, leading to destruction of RGCs and axons. As described above, this mechanism of disease induction is relevant to glaucoma in humans, as significantly higher levels of hsp27- and hsp60-responsive CD4+ T cells were detected in glaucoma patients than age-matched healthy controls. Finally, a transient elevation of IOP is sufficient to induce autoimmune responses, and secondary RGC and axon degeneration, providing an explanation for the continuous disease progression in mice and patients with normal range of IOP and the lack of long-term efficacy by therapies that aim to low the IOP alone. Together, these results demonstrate that activation of CD4+ T cell-mediated autoimmunity plays a profound role and underlies a unifying disease mechanism for pathogenesis of secondary neurodegeneration in the etiology of both high- and normal-tension glaucoma.

The role of hsp in stress-responses and their immunological properties has been explored (Rajaiah, R. & Moudgil, K. D. Autoimmun Rev 8, 388-393 (2009)). Described herein are results that demonstrate that IOP elevation induces hsp27 expression in RGC, which in turn serves a dominant pathogenic autoantigen to stimulate T cell responses in glaucoma. Increased expression of hsp27 in the retina has been noted (Tezel, G., Autoantibodies to small heat shock proteins in glaucoma. Investigative ophthalmology & visual science 39, 2277-2287. (1998); Huang et al., Investigative ophthalmology & visual science 48, 4129-4135. (2007)). Although increased hsp expression can be neuroprotective in the short term (O'Reilly et al., Mol Neurobiol 42, 124-132 (2010); Kelly, S. & Yenari, M. A. Current medical research and opinion 18 Suppl 2, s55-60 (2002)), hsps are highly antigenic and immune-stimulating and may facilitate the initiation and propagation of immune-mediated injury, as seen during the course of arthritis (Rajaiah, R. & Moudgil, Autoimmun Rev 8, 388-393 (2009)). Besides serving as antigens, hsps also enhance immune responses by inducing phagocytosis and processing of chaperoned antigens by dendritic cells. The abilities of hsps to chaperone antigenic peptides or proteins, interact and stimulate antigen presenting cells to secrete inflammatory cytokines, mediate maturation of dendritic cells make them a one-stop shop for inducing immune responses. Furthermore, hsps are conserved between bacteria and human (˜50-70% identity). CD4+ T cells induced by microbial hsps may cross-react with mouse or human hsps, making it easier for TOP to induce hsp-specific CD4+ T cell responses. Nevertheless, mice constitutively overexpressing hsp27 in neurons do not automatically manifest autoimmune disorders or neurodegeneration. This indicates that elevated expression of hsp27 alone is unlikely to evoke autoimmunity, but it may work together with local inflammation or neural damage signals to stimulate T- and B-cell mediated responses. Heat shock proteins are induced under neuronal stress and damage, including trauma and ischemia (Reynolds, L. P. & Allen, G. V. Cerebellum 2, 171-177 (2003)), and may play a wide role in inducing autoimmune responses.

### Advantages

Glaucoma is the most frequent neurodegenerative disorder and a leading cause of blindness worldwide. However, prior to the invention described herein, existing treatments were not effective at controlling the progressive neurodegeneration and vision loss. A lack of reliable and non-invasive biomarkers for early diagnosis and evaluation of treatment efficacy partly contributed to this problem. The findings presented herein indicate that elevated levels of hsp27 or hsp60 specific T cells in patient blood or hsp-specific autoantibodies represent an early diagnostic marker of glaucoma and other ocular neurodegenerative conditions. Moreover, prior to the invention described herein, treatments of glaucoma relied exclusively on lowering TOP. The results presented herein explain their lack of long-term efficacy, and provide an alternate or adjunct method of preventing and treating vision loss by combining TOP lowering drugs with immunosuppressive agents or hsp inhibitors. Identification of a key role of autoreactive CD4+ T cells in glaucoma revealed that preventing and treating the disease is accomplished by modulating these autoreactive T cells.

### Example 2

### Induction of Hsp27 Autoimmunity in Other Forms of Optic Neuropathy and Neuroprotective Effects of Immune Suppressor Rapamycin

The autoimmune responses in other forms of optic neuropathy, including ischemic optic neuropathy and traumatic optic nerve injury (crush injury), were examined. Both ischemic optic neuropathy and optic nerve crush injury induced T cell mediated hsp27 autoimmunity as determined by DTH and ELISPOT assays. These results indicate that autoimmunity is induced widely in several forms of neuronal injury in the optic nerve.

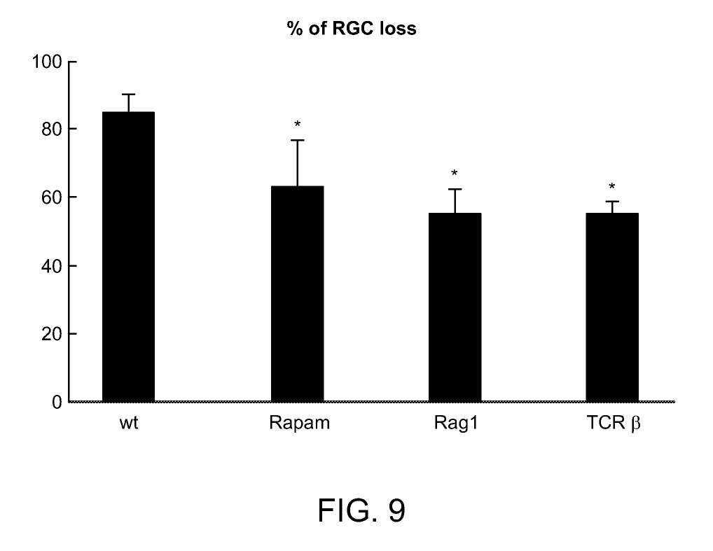

To determine whether blockade of autoimmunity has a benefit effect on neuronal and axon degeneration under various conditions of optic neuropathy, optic nerve crush injury was performed in Rag1−/− and TCRβ−/− mice or wild-type mice that were treated with a general immune suppressor—rapamycin (i.p., 100 μg/day). There was an 87% loss of RGCs at 4 weeks post-optic nerve crush. Mice treated with rapamycin exhibited 65% or 58% and 58% of RGC loss in Rgc1−/− and TCRβ−/−, respectively (FIG. 9). Thus, mice deficient for Rag1 or TCRβ showed significant protective effects for RGCs in optic nerve crush injury models. These results demonstrate that other forms of optic neuropathy also induce autoimmunity specific to hsp27 that can contribute critically to neurodegeneration. These results demonstrate that autoimmunity may be involved widely in many forms of neuronal injury in the optic nerve.

### Example 3

### Ischemic or Stress Insult (Elevated IOP) to the Optic Nerve and Retina Induced a T Cell Response Specific to hsp that Causes Chronic Neurodegeneration

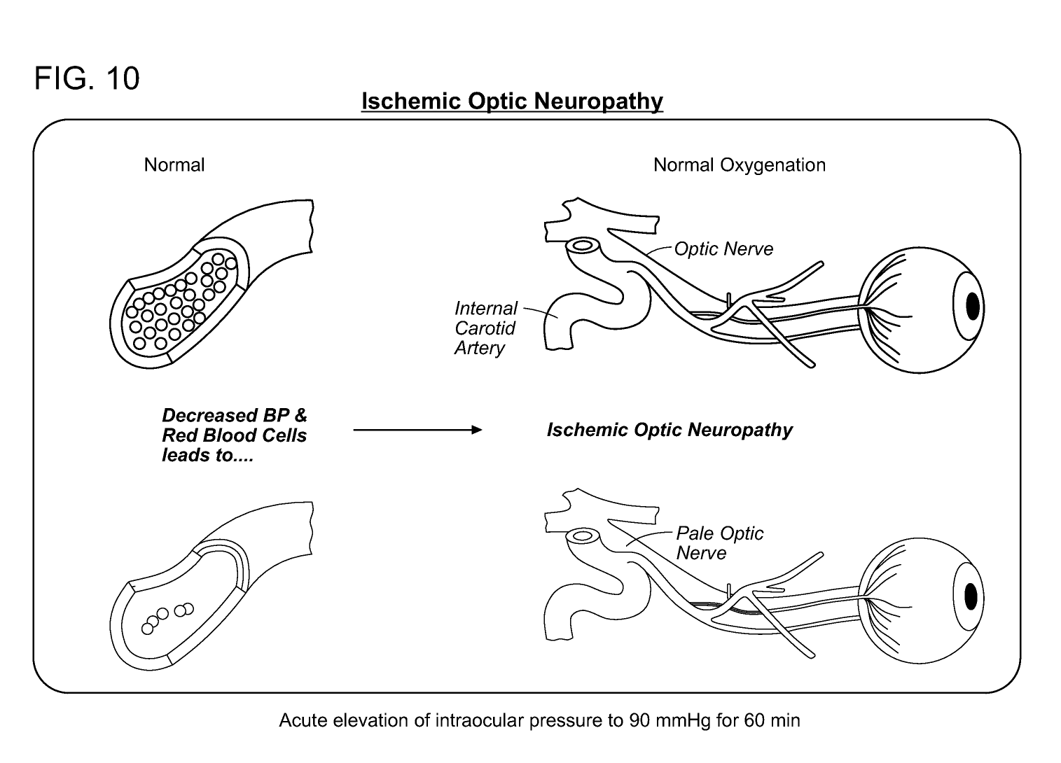

Like glaucoma, AION is an optic nerve disease (FIG. 10). AION results from a sudden ischemic insult to the proximal portion of the optic nerve. AION is the most common cause of sudden optic nerve-related vision loss, and it usually affects individuals over 55 years of age. While typically unilateral, 15-20% of individuals with unilateral AION will experience AION in the contralateral eye over the subsequent 5 years. Prior to the invention described herein, there was no consistently effective treatment, either to improve vision in an eye affected by AION or to prevent visual loss from AION in the fellow eye.

### Acute Ischemic Injury Induced Progressive Neurodegeneration

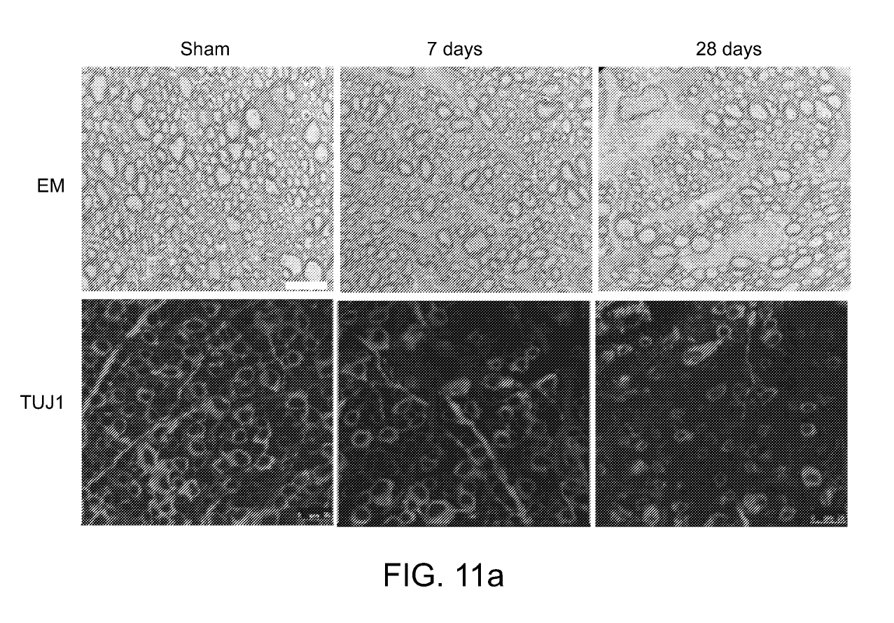

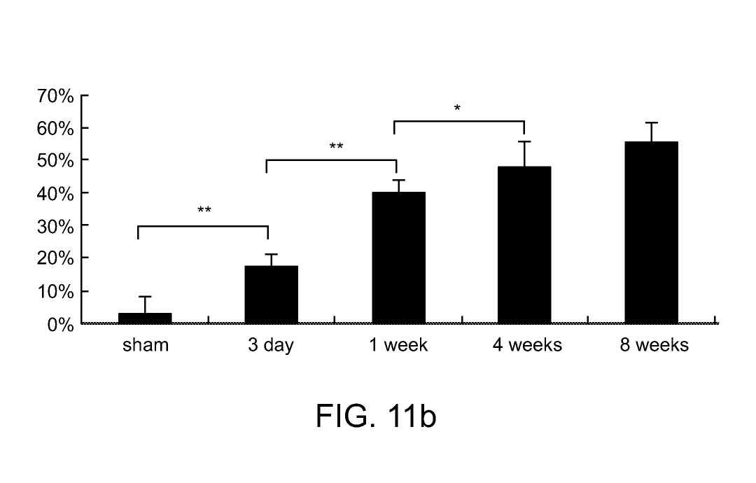

AION in mice induced progressive axon and RGC degeneration that lasted over 4 weeks, which indicates that acute injury triggers a secondary event contributing to the progressive neurodegeneration. FIG. 11A shows representative electron microscopy (EM) and immunofluorescence (Tuj-1; neuron specific antigen) analysis of axon and RGC loss in optic nerve sections and retinal flat-mounts in mice 7 and 28 days following induction of acute AION by elevation of IOP to 100 mmHg for 1 hour. Retinal flat-mounts were immunolabeled with a primary antibody for an RGC specific marker, Tuj 1-1, followed by an AlexaFluor 488-conjugated secondary antibody. Scale bars: 2 μm (EM); 25 μm (Tuj1). FIG. 11B shows the quantification of RGC loss at various time points after the induction of AION. Mice were sacrificed at 0, 3, 7, 28, and 56 days after AION (n=6/group) or at 28 days after sham operation (n=6). Loss of RGCs (mean±S.D.) is presented as percentage of RGC counts from retinal flat-mounts of injured eyes relative to that of the uninjured contralateral eyes. *P<0.05, **P<0.001 by two tailed student t test.

### Induction of Hsp27 and Hsp60 in RGCs Following Ischemic Optic Neuropathy

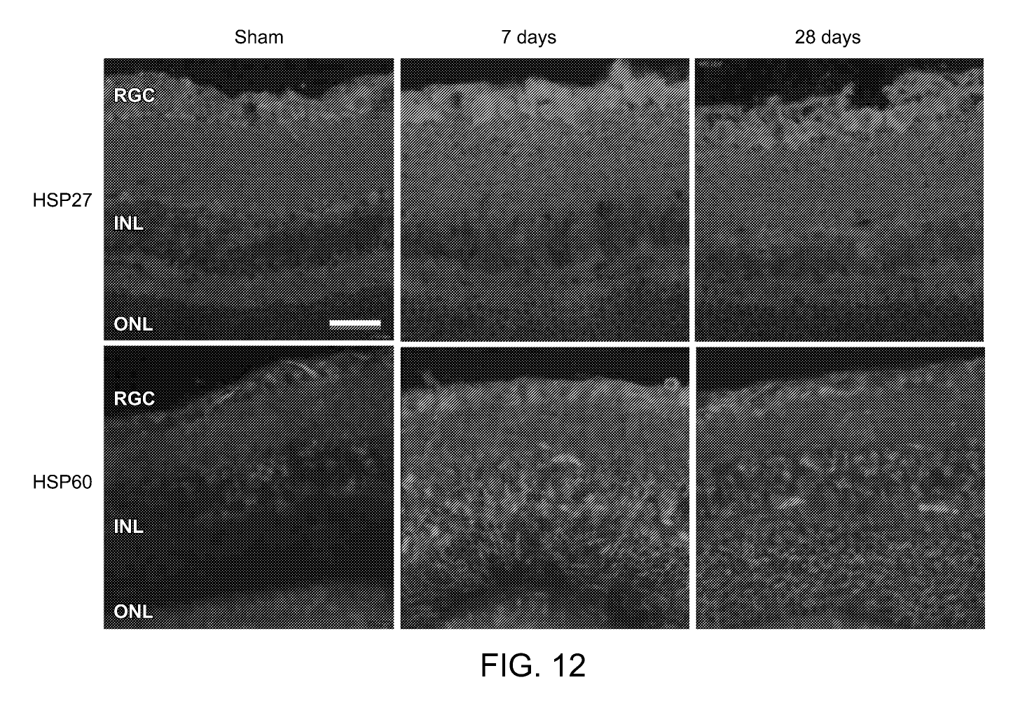

The induction of hsp27 and hsp60 expression in RGCs following AION was analyzed. FIG. 12 shows representative photomicrographs of B6 wild-type mice at 1 and 4 weeks after induction of AION or 4 weeks after sham operation that were immunolabeled for hsp27 and hsp60. These results demonstrate the upregulation of hsp27 and hsp60 in the retina following AION as compared to the wild-type control retina. Scale bar: 15 μm.

T Cell Infiltration into the Retina

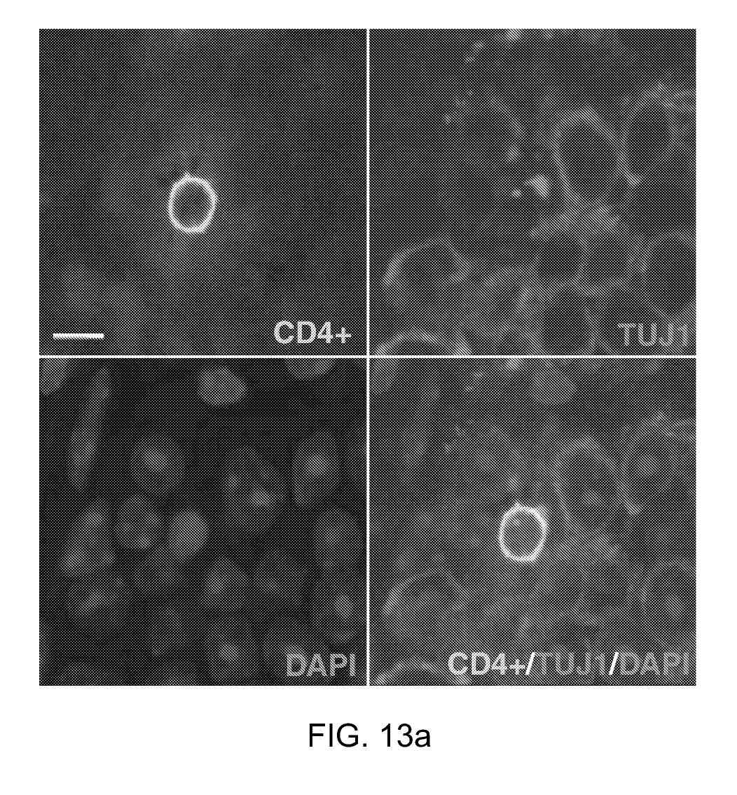

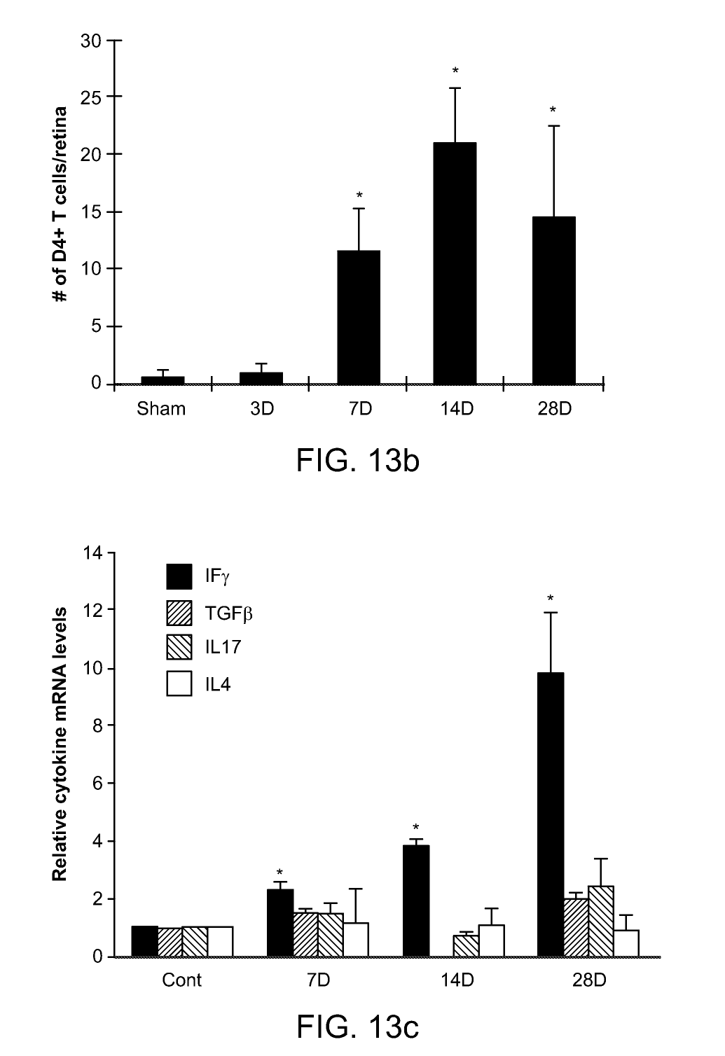

As shown in FIG. 13, AION induces CD4+ T cell infiltration into the retina. FIG. 13A shows double immunolabeling of CD4 (green) and Tuj1 (red) in retinal flat-mounts taken from mice at 2 weeks after the induction of AION. The retina flat-mount was also counter-stained with nuclear marker 4′,6-diamidino-2-phenylindole (DAPI; blue). Scale bar: 10 μm. FIG. 13B shows the quantification of T cell infiltration into retinal flat-mounts of B6 wild-type mice at 3, 7, 14 and 28 days (D) following AION or from 28 days of sham operated mice (n=6/group). *P<0.05 as compared to sham group. FIG. 13C shows the results of RT-PCR that detect 4 types of T cell markers, IFNγ (TH1), interleukin 4 (IL4; TH2), IL17 (TH17) and TNFα (Treg), expression in the injury retina at different time points after AION. The results show a significant increase of IFNγ after AION as compared to sham control, indicating infiltrated T cells are predominantly TH1.

### Ischemic Optic Neuropathy Induced T Cell Responses to Hsp27 and Hsp60

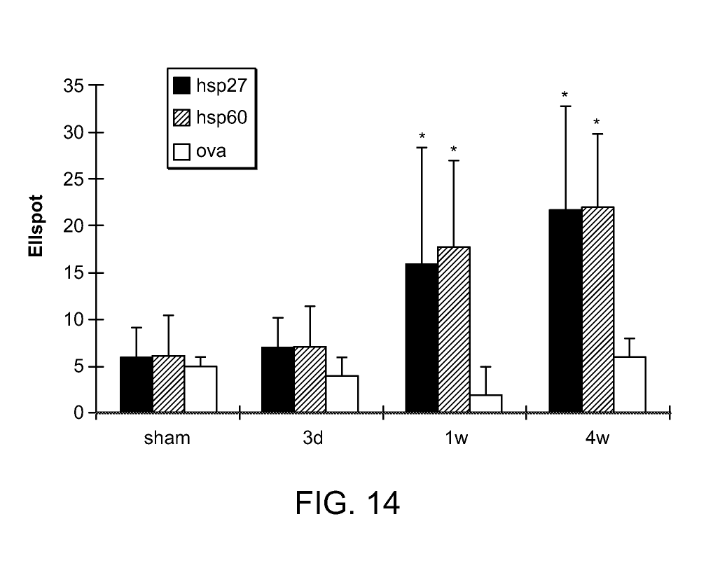

As shown in FIG. 14, acute AION induces hsp27 and hsp60-specific T cell responses. The figure shows quantification of ELISPOT assays that assessed IFN-γ secreting T cells in the lymph node taken from mice at 3, 7 and 28 days after AION. Lymphocytes taken from these mice were stimulated by hsp27, hsp60 or ova (as control stimulation) in vitro. Secretion of IFN-γ was detected by ELISPOT. *P<0.05 as compared to the respective sham groups.

### Increased of IFNγ+ T Cells in the Drainage Lymph Nodes

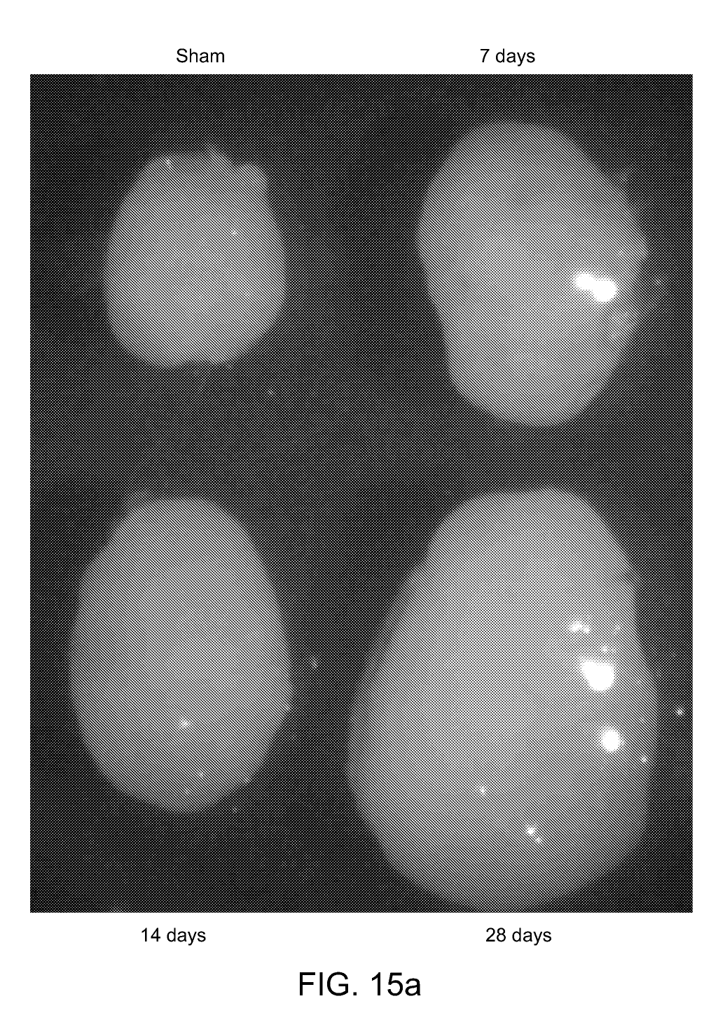

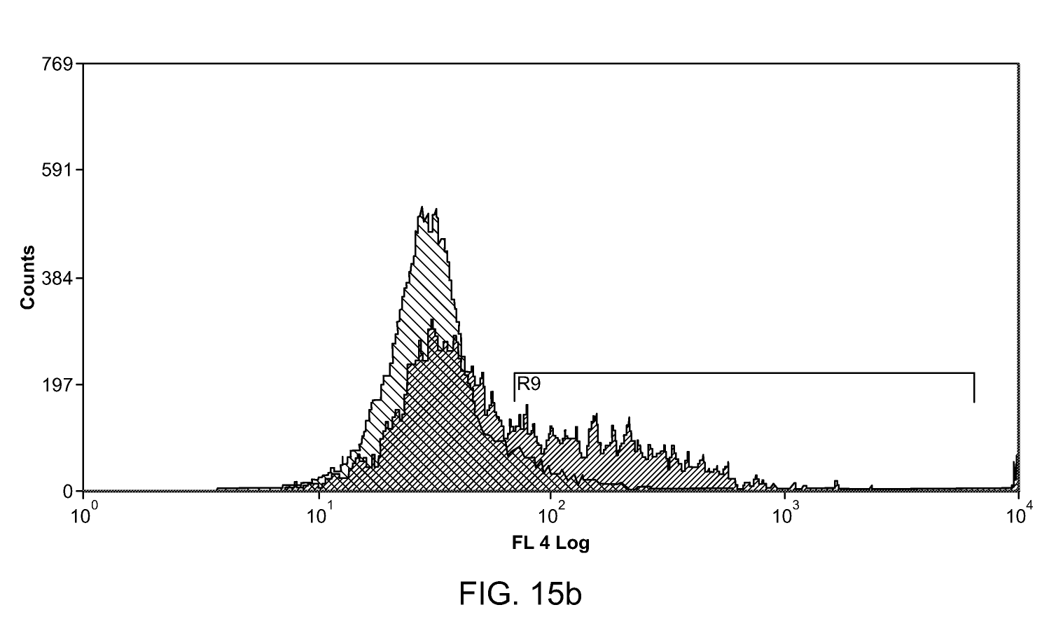

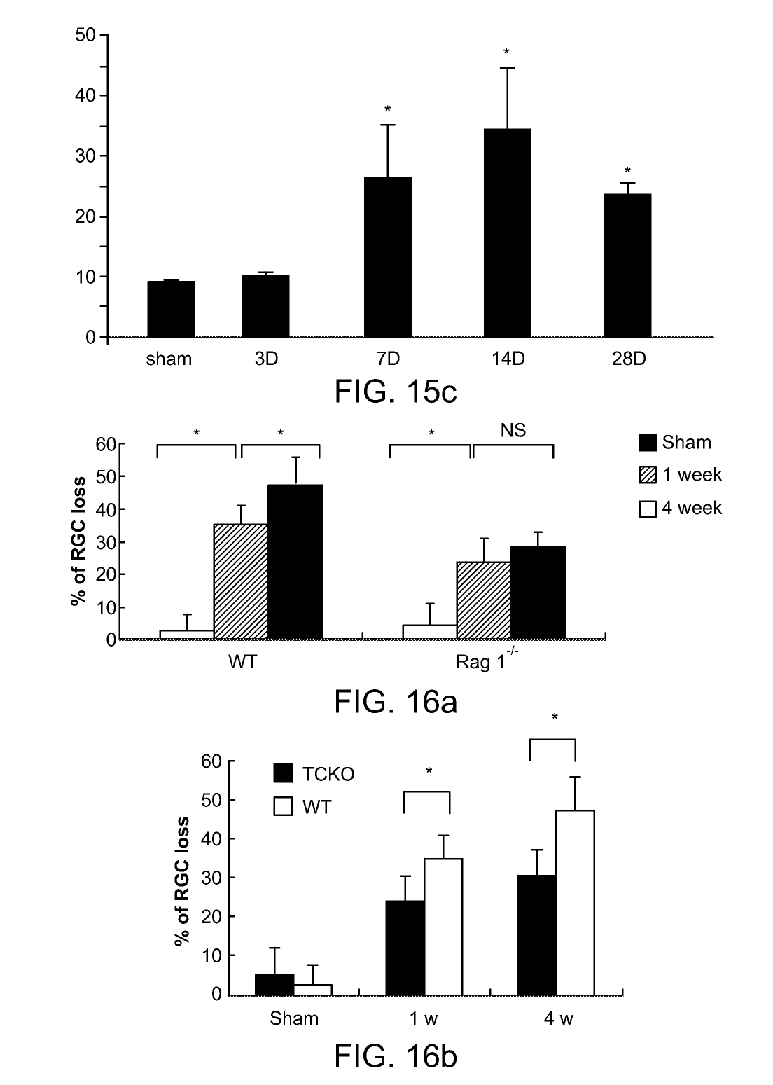

As shown in FIG. 15, acute AION induces CD11b+ cell migration to the draining lymph node and active T cell. FIG. 15A shows representative draining lymph nodes taken from mice at 7, 14 and 28 days post AION induction or from sham-operated group. Representative flow analysis of CD4+/IFNγ+ cells demonstrates an increase of CD4+/IFγ-gamma+ cells in the AION mice as compared to the sham group (FIG. 15B). FIG. 15C shows relative expression of IFNγ+ cells detected at draining lymph node at different time point post AION.

### T Cell Deficiency Attenuated RGC Loss Following Ischemic Optic Neuropathy

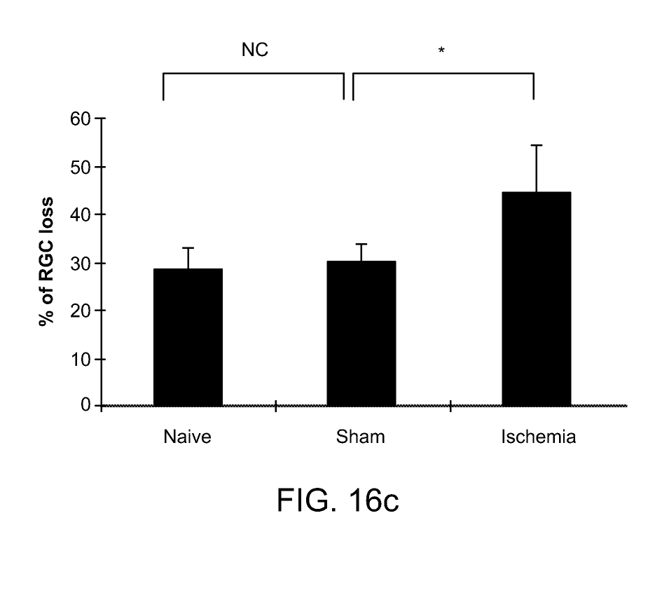

As shown in FIG. 16, T cell deficiency attenuates elevated ischemia-induced secondary axon and RGC degeneration, and transfer of T cells from AION mice restores secondary neurodegeneration in T cell deficient mice. C57BL/6 (B6), Rag1−/− and TCKO mice were induced ischemia and analyzed for axon and RGC loss at 1 and 4 weeks post injury. FIG. 16A shows a comparison of RGC loss (mean±S.D.) among C57BL/6 and Rag1−/− mice between 1 and 4 weeks post ischemia or sham operation at 4 weeks (n=6/group). *P<0.05 N p>0.05. FIG. 16B shows a comparison of RGC loss (mean±S.D.) among C57BL/6 and TCKO mice between 1 and 4 weeks post ischemia or sham operation at 4 weeks (n=6/group). *P<0.05. CD4+ T cells were isolated from the spleen of wild-type ischemia mice and sham group at 2 weeks after injury, and injected into Rag1−/− mice 2 weeks after induction of ischemia. Recipient mice were sacrificed 2 weeks after cell transfer and analyzed for RGCs in retinal flat-mounts. FIG. 16C shows the quantification (mean±S.D.) of RGC loss in Rag1−/− mice that induced ischemia for 4 weeks or received CD4+ T cell transfer from B6 mice with ischemia group or sham group (n=6/group). * indicates p value of <0.05 compared to sham group.

### Example 4

### OKT3 and/or Antibody/Compound that Inhibited T Cell-Mediated Immune Responses was an Effective Therapy for Optic Neuropathy

Muromonab-CD3 antibody (trade name Orthoclone OKT3) is a monoclonal antibody targeted at the CD3 receptor, a membrane protein on the surface of T cells. Muromonab-CD3 antibody is a clinically approved immunosuppressant typically administered to reduce acute rejection in patients with organ transplant. As described in detail below, to determine if Muromonab-CD3 antibody could treat optic neuropathy, Muromonab-CD3 antibody was injected intravitreally at day 3, 7 and 14 after induction of ischemic optic neuropathy.

Other antibodies specific for human CD3 include UCHT1 monoclonal antibody, SK7 monoclonal antibody, and SP7 monoclonal antibody or fragments of such antibodies, so long as they exhibit the desired biological activity. Humanized anti-CD3 antibodies are also useful in the methods of the invention. Humanized antibodies can be ordered from any supplier, e.g., SCL Group.

Anti-T Cell Antibody OKT3 Attenuated RGC Loss after Ischemic Optic Neuropathy

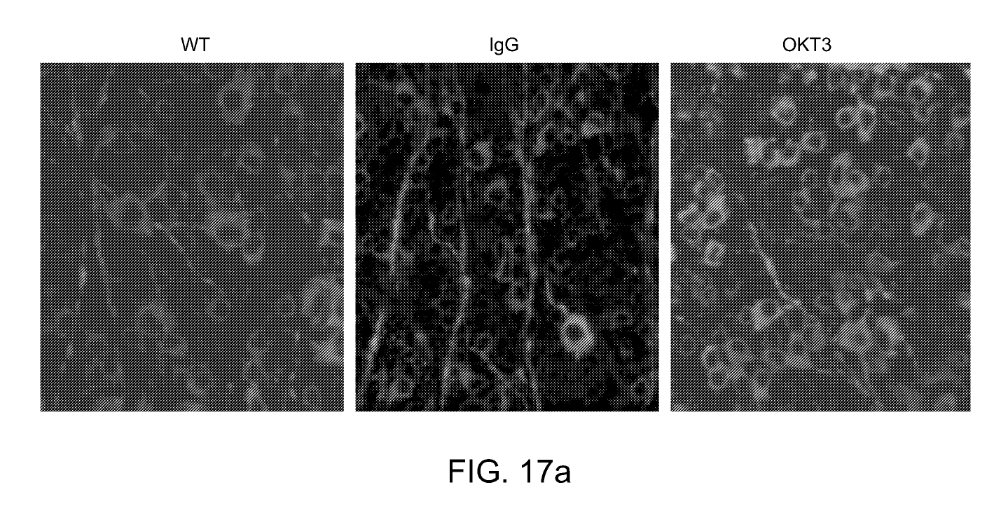

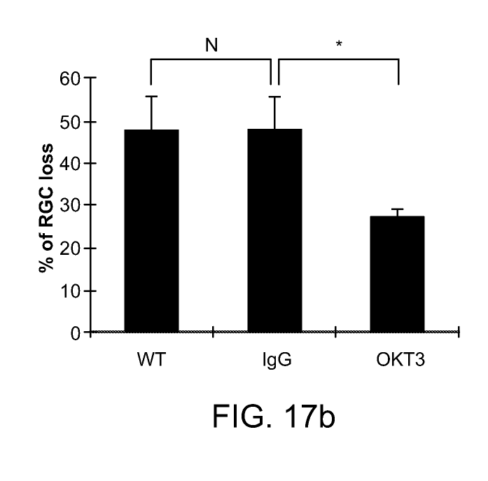

OKT3 antibody administration resulted in a neuroprotective effect for AION (FIG. 17). Briefly, OKT3 antibody was injected into the vitreous of ischemia WT mice at 3, 7, and 14 days post injury. Injection of IgG isotype served as the control. All of the recipient mice were sacrificed at 4 weeks after AION. FIG. 17A shows representative photomicrographs of immunofluorescent labeled RGCs (Tuj-1) in retinal flat-mounts of wild-type mice with AION that received no treatment, control IgG, or OKT3 antibody treatment. FIG. 17B shows the quantification (mean±S.D.) of RGC loss in mice (n=6/group). * indicates p value of <0.05.

Anti-T Cell Antibody OKT3 Rescued Retinal Function after Ischemic Optic Neuropathy

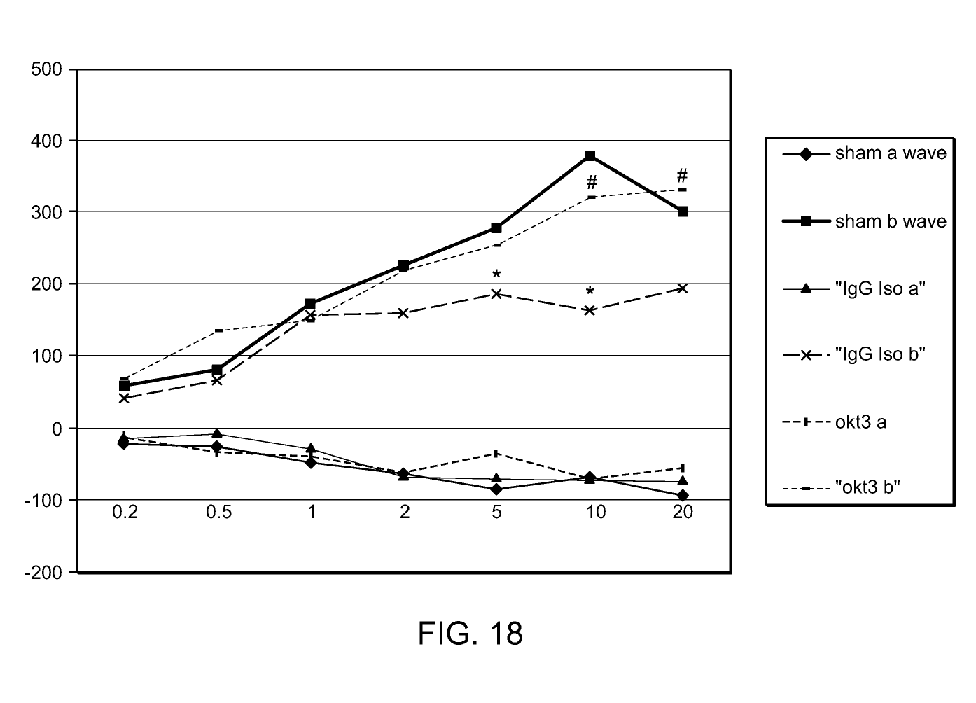

The results presented in FIG. 18 demonstrate the functional rescue of retinal activity by OKT3 treatment after AION. Specifically, the figure shows a comparison of electroretinograma and b wave length (mean±S.D.) between the sham group, IgG isotypeinjected ischemia group and OKT3 antibody injected ischemia group at different intensity of light stimulation. *P<0.05 refers to comparisons between IgG isotype-injected group and sham group. #P<0.05 refers to comparisons between IgG isotype-injected group and OKT3 antibody-injected group (n=6/group).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

We claim:

1. A method for inhibiting or reducing the severity of a heat shock protein (hsp)-mediated ocular neurodegenerative condition in a subject, comprising
identifying a subject characterized as suffering from said condition; and
locally administering to an ocular or adnexal tissue of a subject a composition comprising an immunosuppressant agent;
thereby inhibiting or reducing the severity of said condition.

2. The method of claim 1, wherein said hsp is hsp27 or hsp60.

3. The method of claim 1, wherein said condition is glaucoma, anterior ischemic optic neuropathy (AION), or optic nerve damage.

4. The method of claim 1, wherein said immunosuppressive agent is an antibody, a small molecule, a glucocorticoid, a cytostatic, an inhibitor of hsp27, an inhibitor of hsp60, cyclosporine, FK506, tacrolimus, rapamycin, an interferon, an opiod, tumor necrosis factor-alpha binding protein, mycophenolate, fingolimod, or myriocin.

5. The method of claim 4, wherein said antibody is an antibody specific for CD3.

6. The method of claim 5, wherein said antibody specific for CD3 is a monoclonal antibody specific for human CD3.

7. The method of claim 1, wherein said subject has elevated intraocular pressure.

8. The method of claim 1, wherein said subject has normal intraocular pressure with optic nerve cupping and visual field loss characteristic of glaucoma.

9. The method of claim 1, wherein said method comprises inhibiting or reducing the severity of secondary phase neuronal damage.

10. The method of claim 1, wherein said method comprises inhibiting or reducing the severity of retinal ganglion cell (RGC) damage or axonal damage.

11. The method of claim 3, wherein said glaucoma is primary open angle glaucoma, closed angle glaucoma, secondary glaucoma, or congenital glaucoma.

12. The method of claim 1, further comprising administering an inhibitor of T cell or B cell-mediated autoimmunity.

13. The method of claim 12, wherein said inhibitor of T cell-mediated autoimmunity is an inhibitor of CD4+ T cell-mediated autoimmunity to hsp27 or hsp60.

14. The method of claim 12, wherein said inhibitor of T cell-mediated autoimmunity is dantrolene, FUT-175, a Kv1.3 inhibitor, a phosphodiesterase-3 inhibitor, a phosphodiesterase-4 inhibitor, an antibody that depletes T cells, or a molecule that suppresses T cell function without eliminating T cells.

15. The method of claim 14, wherein said antibody that depletes T cells is an anti-CD3 antibody, an anti-CD4 antibody, or an anti-CD52 antibody.

16. The method of claim 1, further comprising administering an agent that reduces intraocular pressure.

17. The method of claim 16, wherein said agent that reduces intraocular pressure is selected from the group consisting of pilocarpine, timolol, acetazolamide, clonidine, ecothiopate, carteolol, dorzolamide, apraclonidine, latanoprost, and bimatoprost.

18. The method of claim 1, wherein said immunosuppressant agent comprises a polynucleotide, a polypeptide, an antibody, or a small molecule.

19. The method of claim 1, wherein the form of said composition is a solid, a paste, an ointment, a gel, a liquid, an aerosol, a mist, a polymer, a film, an emulsion, or a suspension.

20. The method of claim 1, wherein said composition is administered topically.

21. The method of claim 1, wherein said identifying step comprises detection of a sign or symptom selected from the group consisting of loss of peripheral vision, optic nerve cupping, thinning of the nerve fiber layer, severe unilateral eye pain, cloudy vision, nausea and vomiting, red eye, swollen eye, eye enlargement, light sensitivity, and tearing.

22. A method of diagnosing an hsp-mediated ocular neurodegenerative condition in a subject comprising:
providing a test sample from a subject;
detecting auto-antigen antibodies or auto-antigen-specific T cells in said test sample;
comparing the levels of said auto-antigen antibodies or said auto-antigen-specific T cells in said test sample to a control level of said antibodies or T cells, wherein a higher level of said antibodies or T cells compared to said control level is indicative of said condition;
thereby diagnosing condition in said subject.

23. The method of claim 22, wherein said condition is glaucoma, anterior ischemic optic neuropathy (AION), or optic nerve damage.

24. The method of claim 22, wherein said subject comprises RGC damage or axonal damage.

25. The method of claim 22, wherein said test sample is obtained from a biological fluid selected from the group consisting of whole blood, serum, plasma, vitreous humor, and aqueous humor.

26. The method of claim 22, wherein said auto-antigen is selected from the group consisting of hsp-27, hsp-60, alpha-A-crystallin, and alpha-B-crystallin.

27. The method of claim 22, wherein said control level is obtained from age-matched healthy individuals.

28. The method of claim 23, wherein said glaucoma is diagnosed prior to vision impairment.

29. A method for inhibiting or reducing the severity of optic neuropathy, comprising
identifying a subject characterized as suffering from ischemia or trauma-induced optic neuropathy; and
locally administering to an ocular or adnexal tissue of a subject an immunosuppressant agent;
thereby inhibiting or reducing the severity of optic neuropathy.

30. The method of claim 29, wherein said method comprises inhibiting or reducing the severity of secondary phase neuronal damage associated with optic neuropathy.

31. The method of claim 29, wherein said optic neuropathy is anterior ischemic optic neuropathy (AION).

32. The method of claim 29, wherein said method comprises inhibiting or reducing the severity of retinal ganglion cell (RGC) damage or axonal damage.

33. The method of claim 29, wherein said immunosuppressant agent is muromonuab-CD3 antibody OKT3.

34. The method of claim 29, further comprising administering an inhibitor of T cell or B cell-mediated autoimmunity.

35. The method of claim 29, wherein said inhibitor of T cell-mediated autoimmunity is an inhibitor of CD4+ T cell-mediated autoimmunity to heat shock protein 27 (hsp27) or hsp60.

36. The method of claim 29, further comprising administering an agent that reduces intraocular pressure.

37. The method of claim 29, further comprising administering an inhibitor of hsp27 or hsp60.

38. A method of diagnosing optic neuropathy in a subject comprising;
providing a test sample from a subject;
detecting auto-antigen antibodies or auto-antigen-specific T cells in said test sample;
comparing the levels of said auto-antigen antibodies or said auto-antigen-specific T cells in said test sample to a control level of said antibodies or T cells, wherein a higher level of said antibodies or T cells compared to said control level is indicative of optic neuropathy;
thereby diagnosing optic neuropathy in said subject.

39. The method of claim 38, wherein said subject comprises RGC damage or axonal damage.

40. The method of claim 38, wherein said test sample is obtained from a biological fluid selected from the group consisting of whole blood, serum, plasma, vitreous humor, and aqueous humor.

41. The method of claim 38, wherein said auto-antigen is selected from the group consisting of hsp-27, hsp-60, alpha-A-crystallin, and alpha-B-crystallin.

42. The method of claim 38, wherein said optic neuropathy is anterior ischemic optic neuropathy (AION).
